(19) United States
(12) Patent Application Publication
(10) Pub. No.: US 2026/0276655 A1
(43) Pub. Date: Sep. 17, 2026

(54) BIOMARKERS OF AMYOTROPHIC LATERAL SCLEROSIS AND USES THEREOF

(71) Applicants: The Methodist Hospital, Houston, TX (US); Coya Therapeutics, Inc., Houston, TX (US)

(72) Inventors: Stanley Hersh APPEL, Houston, TX (US); David Robert BEERS, Houston, TX (US); Howard BERMAN, Houston, TX (US)

(21) Appl. No.: 19/166,854

(22) PCT Filed: Mar. 20, 2024
(86) PCT No.: PCT/US2024/020626

§ 371 Date: Sep. 18, 2025

Related U.S. Application Data
(60) Provisional application No. 63/453,686, filed on Mar. 21, 2023.

(51) Int. Cl. G01N 33/68; G01N 33/543; G01N 33/92

(52) U.S. Cl. CPC G01N 33/6896; G01N 33/543; G01N 33/6869; G01N 33/92; G01N 2333/523; G01N 2333/54; G01N 2333/775; G01N 2800/2821; G01N 2800/52

(57) ABSTRACT

The present disclosure relates to biomarkers and uses thereof in methods for selecting a patient diagnosed with amyotrophic lateral sclerosis (ALS) for an ALS therapy. The present disclosure further relates to methods for identifying the severity of ALS in a patient, treating an ALS patient, and monitoring efficacy of an ALS treatment.

### 1. CROSS-REFERENCE TO RELATED APPLICATIONS

This application claims the benefit of U.S. Provisional Patent Application No. 63/453,686, filed Mar. 21, 2023, which is incorporated by reference herein in its entirety.

### 2. FIELD

Provided herein are biomarkers and uses thereof in methods for selecting a patient diagnosed with amyotrophic lateral sclerosis (ALS) for an ALS therapy. Methods for identifying the severity of ALS in a patient, treating an ALS patient, and monitoring efficacy of treatment, are also provided herein.

### 3. BACKGROUND

Amyotrophic lateral sclerosis (ALS) is the most common form of motor neuron disease resulting from neurodegeneration, which leads to progressive loss of motor neurons. ALS is characterized by heterogeneous clinical presentations and rates of progression among affected individuals. Given the variation in ALS disease from patient to patient, monitoring disease progression in ALS using the currently available tools is subjective at best.

Currently, ALS remains a fatal disease as there is no cure for ALS. There is also evidence that the condition is increasing in incidence. As such, there is a need in the field to identify and develop more effective and beneficial treatments and regimens for ALS patients.

However, identifying new therapies to halt or reverse the progress of the disease and tracking the effectiveness of therapies requires an objective and reliable method for monitoring ALS progression and, ultimately, evaluating the effect of therapies. Almost all ALS clinical trials have employed the ALS Functional Rating Scale-Revised (ALSFRS-R) as the primary outcome measure for assessing ALS disease progression and functional disability; however, the linearity of the measurement varies over time, while the heterogeneous nature of the disease affects ALSFRS-R results between ALS clinical subgroups. Therefore, the development of objective measures of functional disability in ALS that accurately reflect patients' disease burdens, progression rates, and responsiveness to treatments is needed in ALS clinical trials and beyond.

### 4. SUMMARY

In one aspect, the present disclosure provides a method of treating amyotrophic lateral sclerosis (ALS), the method comprising: (a) collecting a serum sample from the patient before administering a first ALS therapy; (b) administering the first ALS therapy to a patient diagnosed with ALS; (c) collecting a serum sample from the patient after administering the first ALS therapy; (d) measuring concentration of at least one biomarker in the serum samples collected from the patient before and after administering the first ALS therapy, wherein the at least one biomarker is selected from the group consisting of oxidized low-density lipoprotein receptor 1 (OLR1), oxidized low-density lipoprotein (ox-LDL), 4-hydroxynonenal (4-HNE), soluble CD14 (sCD14), lipopolysaccharide binding protein (LBP), C-reactive protein (CRP), interleukin 17F (IL-17F), interleukin 17C (IL-17C), monocyte chemoattractant protein-1 MCP-1 (CCL2), and interleukin-18 (IL-18); (e) comparing the concentration of the at least one biomarker in the serum sample collected from the patient after administering the first ALS therapy to a baseline, wherein the concentration of the at least one biomarker in the serum sample collected from the patient before administering the first ALS therapy is the baseline; and (f) administering a second ALS therapy to the patient if the concentration of at least one biomarker after administering the first ALS therapy is at least one-fold different than the baseline.

In certain embodiments, the method of treating ALS further comprises collecting serum samples on a serial basis for the duration of the ALS therapy, wherein the serum samples are collected at least once a week, at least once every two weeks, or at least once a month.

In certain embodiments, the second ALS therapy is administered to the patient if the concentration of at least 2, at least 3, at least 4, or at least 5 biomarkers are at least one-fold different than the baseline. In some embodiments, the second ALS therapy is administered to the patient if the concentration of at least one biomarker is at least one-fold higher than the baseline. In some embodiments, the second ALS therapy is administered to the patient if the concentration of at least one biomarker is at least one-fold less than the baseline. In some embodiments, the second ALS therapy is administered to the patient if the concentration of at least one biomarker is at least one-fold higher than the baseline and at least one additional biomarker is at least one-fold lower than the baseline. In certain embodiments, the second ALS therapy is administered to the patient if the concentration of CCL2, IL-18, or both are at least one-fold lower than the baseline.

In certain embodiments, the second ALS therapy administered to the patient is the same as the first ALS therapy administered. In certain embodiments, the second ALS therapy is not administered to the patient if the concentration of CCL2, IL-18, or both are at least one-fold higher than the baseline. In certain embodiments, the second ALS therapy administered to the patient is not the same as the first ALS therapy administered if the concentration of CCL2, IL-18, or both are at least one-fold higher than the baseline.

In certain embodiments, the serum sample is collected from the patient at least 1 day, at least 3 days, at least 5 days, at least 1 week, at least 2 weeks, at least 3 weeks, or at least after 4 weeks of administering the first ALS therapy.

In certain embodiments, the first ALS therapy is the same as the second ALS therapy administered. In certain embodiments, the first ALS therapy is different from the second ALS therapy administered.

In certain embodiments, the ALS therapy comprises one or more agents that targets inflammation and/or oxidative stress. In certain embodiments, the ALS therapy comprises at least one gene therapy, wherein the gene therapy targets one or more mutations in genes SOD1, C9orf72, FUS, or any combination thereof. In certain embodiments, the ALS therapy comprises a Treg infusion. In certain embodiments, the ALS therapy comprises Relyvrio™ (AMX0035), edaravone, riluzole, or any combination thereof. In certain embodiments, the ALS therapy comprises IL-2. In certain embodiments, the ALS therapy comprises IL-2 and abatacept (CTLA-4 Ig (e.g., ORENCIA®)). In certain embodiments, the ALS therapy comprises an anti-inflammatory agent. In certain embodiments, the ALS therapy comprises an antioxidant agent.

In certain embodiments, the second ALS therapy comprises an anti-inflammatory agent if the concentration of CCL2, IL-18, or both are at least one-fold higher than the baseline.

In certain embodiments, the second ALS therapy comprises an anti-antioxidant agent if the concentration of ox-LDL, OLR1, or both are at least one-fold higher than the baseline.

In certain embodiments, the method further comprises determining an Appel ALS clinical score (AALS) for the patient before and after administering the first ALS therapy. In some embodiments, the second ALS therapy is administered to the patient if the concentration of at least one biomarker is at least one-fold different than the baseline and the AALS score for the patient is the same or lower than the AALS score for the patient before administering the first ALS therapy.

In certain embodiments, the concentration of the at least one biomarker is determined by enzyme linked-immunosorbent assay (ELISA), protein immunoprecipitation, immunoelectrophoresis, chemical analysis, SDS-PAGE and Western blot analysis, protein immunostaining, electrophoresis analysis, competitive binding assay, functional protein assay, protein microarray, high-performance liquid chromatography (HPLC), mass spectrometry, liquid chromatography-mass spectrometry (LC/MS), capillary electrophoresis (CE)-MS, cytometric bead array analysis, immunonephelometric assays, fluorescence activated cell sorting (FACS) analysis, microscopic analysis, microarray, or any combination thereof.

In another aspect, the present disclosure provides a method for selecting a patient for ALS therapy, the method comprising: (a) determining whether a concentration of at least one biomarker in a serum sample collected from a patient diagnosed with or suspected of having ALS is less than, equal to, or greater than, a reference concentration; (b) wherein the at least one biomarker is selected from the group consisting of oxidized low-density lipoprotein receptor 1 (OLR1), oxidized low-density lipoprotein (ox-LDL), monocyte chemoattractant protein-1 MCP-1 (CCL2), interleukin-18 (IL-18), and any combination thereof; and, (c) selecting the patient for ALS therapy if at least one biomarker is equal to or less than the reference concentration.

In certain embodiments, the method for selecting a patient for ALS therapy further comprises administering the ALS therapy to the selected patient.

In certain embodiments, the ALS therapy comprises Relyvrio™ (AMX0035), edaravone, riluzole, Treg infusion, IL-2, or any combination thereof.

In certain embodiments, the patient is selected for ALS therapy if at least two or more biomarkers are equal to or less than the reference concentration. In some embodiments, the at least two or more biomarkers are CCL2 and IL-18.

In certain embodiments, the patient is naïve to ALS therapy. In certain embodiments, the patient has previously received at least one ALS therapy.

In certain embodiments, the patient has received at least one ALS therapy and is selected for a subsequent administration of the same ALS therapy if at least one biomarker is equal to or less than the reference concentration. In some embodiments, the ALS therapy is a Treg infusion, IL-2, or a combination thereof.

In certain embodiments, the reference concentration is an average concentration of the biomarker measured in serum samples collected from at least 10 healthy subjects having the same age, gender, and/or ethnicity as the patient.

In certain embodiments, the concentration of the at least one biomarker is determined by enzyme linked-immunosorbent assay (ELISA), protein immunoprecipitation, immunoelectrophoresis, chemical analysis, SDS-PAGE and Western blot analysis, protein immunostaining, electrophoresis analysis, competitive binding assay, functional protein assay, protein microarray, high-performance liquid chromatography (HPLC), mass spectrometry, liquid chromatography-mass spectrometry (LC/MS), capillary electrophoresis (CE)-MS, cytometric bead array analysis, immunonephelometric assays, fluorescence activated cell sorting (FACS) analysis, microscopic analysis, microarray, or any combination thereof.

In another aspect, the present disclosure provides a kit for detecting and quantifying the level of one or more ALS biomarkers in a serum sample obtained from a patient having or suspected of having ALS, wherein the ALS biomarkers are selected from the group consisting of oxidized low-density lipoprotein receptor 1 (OLR1), oxidized low-density lipoprotein (ox-LDL), monocyte chemoattractant protein-1 MCP-1 (CCL2), interleukin-18 (IL-18), and any combination thereof.

In certain embodiments, the kit comprises a solid surface having an antibody or a fragment thereof comprising a binding epitope for the ALS biomarkers OLR1, ox-LDL, IL-17C, CCL2, IL-18, or any combination thereof, and at least one reagent for detecting formation of a biomarker-antibody complex.

In certain embodiments, the kit is for use in selecting a patient for an ALS therapy. In certain embodiments, the kit is for use in monitoring effectiveness of an ALS therapy, wherein effectiveness can be assessed during the course of or after completing an ALS therapy regimen.

In another aspect, the present disclosure provides a method of treating treatment-responsive ALS, comprising administering to a patient diagnosed with treatment-responsive ALS a therapeutically effective amount of an ALS therapy, wherein the treatment-responsive ALS is characterized as having at least one biomarker equal to or less than a reference concentration, wherein the at least one biomarker is selected from the group consisting of oxidized low-density lipoprotein receptor 1 (OLR1), oxidized low-density lipoprotein (ox-LDL), monocyte chemoattractant protein-1MCP-1 (CCL2), and interleukin-18 (IL-18); and, wherein the reference concentration is an average concentration of the biomarker measured in serum samples collected from at least 10 healthy subjects having the same age, gender, and/or ethnicity as the patient.

In certain embodiments, treatment-responsive ALS is characterized as having at least two biomarkers equal to or less than a reference concentration. In some embodiments, the at least two or more biomarkers are CCL2 and IL-18.

In certain embodiments, the ALS therapy comprises Relyvrio™ (AMX0035), edaravone, riluzole, Treg infusion, IL-2, or any combination thereof. In some embodiments, the ALS therapy is a Treg infusion, IL-2, or a combination thereof.

In another aspect, the present disclosure provides a method of determining whether a patient diagnosed with ALS is indicated as likely to be responsive to ALS treatment, the method comprising detecting the concentration of at least one biomarker compared to a reference concentration in a serum sample collected from the patient, wherein the at least one biomarker having a concentration equal to or less than the reference concentration indicates that the patient is likely to be responsive to ALS treatment, wherein the at least one biomarker is selected from the group consisting of oxidized low-density lipoprotein receptor 1 (OLR1), oxidized low-density lipoprotein (ox-LDL), monocyte chemoattractant protein-1 MCP-1 (CCL2), and interleukin-18 (IL-18); and, wherein the reference concentration is an average concentration of the biomarker measured in serum samples collected from at least 10 healthy subjects having the same age, gender, and/or ethnicity as the patient.

In certain embodiments, at least two biomarkers having a concentration equal to or less than a reference concentration indicates that the patient is likely to be responsive to ALS treatment. In some embodiments, the at least two or more biomarkers are CCL2 and IL-18.

In certain embodiments, the ALS therapy comprises Relyvrio™ (AMX0035), edaravone, riluzole, Treg infusion, IL-2, or any combination thereof. In some embodiments, the ALS therapy is a Treg infusion, IL-2, or a combination thereof.

### 4.1 Illustrative Embodiments

Illustrative embodiments of the present disclosure are provided in the paragraphs below:
1. A method of treating amyotrophic lateral sclerosis (ALS) comprising:
   (a) collecting a serum sample from the patient before administering a first ALS therapy;
   (b) administering the first ALS therapy to a patient diagnosed with ALS;
   (c) collecting a serum sample from the patient after administering the first ALS therapy;
   (d) measuring concentration of at least one biomarker in the serum samples collected from the patient before and after administering the first ALS therapy, wherein the at least one biomarker is selected from the group consisting of oxidized low-density lipoprotein receptor 1 (OLR1), oxidized low-density lipoprotein (ox-LDL), 4-hydroxynonenal (4-HNE), soluble CD14 (sCD14), lipopolysaccharide binding protein (LBP), C-reactive protein (CRP), interleukin 17F (IL-17F), interleukin 17C (IL-17C), monocyte chemoattractant protein-1 MCP-1 (CCL2), and interleukin-18 (IL-18);
   (e) comparing the concentration of the at least one biomarker in the serum sample collected from the patient after administering the first ALS therapy to a baseline, wherein the concentration of the at least one biomarker in the serum sample collected from the patient before administering the first ALS therapy is the baseline; and
   (f) administering a second ALS therapy to the patient if the concentration of at least one biomarker after administering the first ALS therapy is at least one-fold different than the baseline.
2. The method of embodiment 1 further comprising collecting serum samples on a serial basis for the duration of the ALS therapy, wherein the serum samples are collected at least once a week, at least once every two weeks, or at least once a month.
3. The method of embodiment 1 or embodiment 2, wherein the second ALS therapy is administered to the patient if the concentration of at least 2, at least 3, at least 4, or at least 5 biomarkers are at least one-fold different than the baseline.
4. The method of any one of embodiments 1-3, wherein the second ALS therapy is administered to the patient if the concentration of at least one biomarker is at least one-fold higher than the baseline.
5. The method of any one of embodiments 1-4, wherein the second ALS therapy is administered to the patient if the concentration of at least one biomarker is at least one-fold less than the baseline.
6. The method of any one of embodiments 1-5, wherein the second ALS therapy is administered to the patient if the concentration of at least one biomarker is at least one-fold higher than the baseline and at least one additional biomarker is at least one-fold lower than the baseline.
7. The method of any one of embodiments 1-6, wherein the second ALS therapy is administered to the patient if the concentration of CCL2, IL-18, or both are at least one-fold lower than the baseline.
8. The method of embodiment 7, wherein the second ALS therapy administered to the patient is the same as the first ALS therapy administered.
9. The method of any one of embodiments 1-6, wherein the second ALS therapy is not administered to the patient if the concentration of CCL2, IL-18, or both are at least one-fold higher than the baseline.
10. The method of any one of embodiments 5-8, wherein the second ALS therapy administered to the patient is not the same as the first ALS therapy administered if the concentration of CCL2, IL-18, or both are at least one-fold higher than the baseline.
11. The method of any one of embodiments 1-10, wherein the serum sample is collected from the patient at least 1 day, at least 3 days, at least 5 days, at least 1 week, at least 2 weeks, at least 3 weeks, or at least after 4 weeks of administering the first ALS therapy.
12. The method of any one of embodiments 1-11, wherein the first ALS therapy is the same as the second ALS therapy administered.
13. The method of any one of embodiments 1-11, wherein the first ALS therapy is different from the second ALS therapy administered.
14. The method of any one of embodiments 1-13, wherein the ALS therapy comprises one or more agents that targets inflammation and/or oxidative stress.
15. The method of any one of embodiments 1-14, wherein the ALS therapy comprises at least one gene therapy, wherein the gene therapy targets one or more mutations in genes SOD1, C9orf72, FUS, or any combination thereof.
16. The method of any one of embodiments 1-15, wherein the ALS therapy comprises a Treg infusion.
17. The method of any one of embodiments 1-16, wherein the ALS therapy comprises Relyvrio™ (AMX0035), edaravone, riluzole, or any combination thereof.
18. The method of any one of embodiments 1-17, wherein the ALS therapy comprises IL-2.
19. The method of any one of embodiments 1-18, wherein the ALS therapy comprises IL-2 and abatacept (CTLA-4 Ig).
20. The method of any one of embodiments 1-19, wherein the ALS therapy comprises an anti-inflammatory agent.
21. The method of any one of embodiments 1-20, wherein the ALS therapy comprises an antioxidant agent.
22. The method of any one of embodiments 1-21, wherein the second ALS therapy comprises an anti-inflammatory agent if the concentration of CCL2, IL-18, or both are at least one-fold higher than the baseline.
23. The method of any one of embodiments 1-22, wherein the second ALS therapy comprises an anti-antioxidant agent if the concentration of ox-LDL, OLR1, or both are at least one-fold higher than the baseline.
24. The method of any one of embodiments 1-23, wherein the method further comprises determining an Appel ALS clinical score (AALS) for the patient before and after administering the first ALS therapy.
25. The method of embodiment 24, wherein the second ALS therapy is administered to the patient if the concentration of at least one biomarker is at least one-fold different than the baseline and the AALS score for the patient the same or lower than the AALS score for the patient before administering the first ALS therapy.
26. The method of any one of embodiments 1-25, wherein the concentration of the at least one biomarker is determined by enzyme linked-immunosorbent assay (ELISA), protein immunoprecipitation, immunoelectrophoresis, chemical analysis, SDS-PAGE and Western blot analysis, protein immunostaining, electrophoresis analysis, competitive binding assay, functional protein assay, protein microarray, high-performance liquid chromatography (HPLC), mass spectrometry, liquid chromatography-mass spectrometry (LC/MS), capillary electrophoresis (CE)-MS, cytometric bead array analysis, immunonephelometric assays, fluorescence activated cell sorting (FACS) analysis, microscopic analysis, microarray, or any combination thereof.
27. A method for selecting a patient for amyotrophic lateral sclerosis (ALS) therapy, the method comprising:
   (a) determining whether a concentration of at least one biomarker in a serum sample collected from a patient diagnosed with or suspected of having ALS is less than, equal to, or greater than, a reference concentration;
   (b) wherein the at least one biomarker is selected from the group consisting of oxidized low-density lipoprotein receptor 1 (OLR1), oxidized low-density lipoprotein (ox-LDL), monocyte chemoattractant protein-1 MCP-1 (CCL2), interleukin-18 (IL-18), and any combination thereof; and,
   (c) selecting the patient for ALS therapy if at least one biomarker is equal to or less than the reference concentration.
28. The method of embodiment 27, further comprising administering the ALS therapy to the selected patient.
29. The method of embodiment 27 or embodiment 28, wherein the ALS therapy comprises Relyvrio™ (AMX0035), edaravone, riluzole, Treg infusion, IL-2, or any combination thereof.
30. The method of any one of embodiments 27-29, wherein the patient is selected for ALS therapy if at least two or more biomarkers are equal to or less than the reference concentration.
31. The method of embodiment 30, wherein the at least two or more biomarkers are CCL2 and IL-18.
32. The method of any one of embodiments 27-31, wherein the patient is naïve to ALS therapy.
33. The method of any one of embodiments 27-32, wherein the patient has previously received at least one ALS therapy.
34. The method of any one of embodiments 27-33, wherein the patient has received at least one ALS therapy and is selected for a subsequent administration of the same ALS therapy if at least one biomarker is equal to or less than the reference concentration.
35. The method of embodiment 34, wherein the ALS therapy is a Treg infusion, IL-2, or a combination thereof.
36. The method of any one of embodiments 27-35, wherein the reference concentration is an average concentration of the biomarker measured in serum samples collected from at least 10 healthy subjects having the same age, gender, and ethnicity as the patient.
37. The method of any one of embodiments 27-36, wherein the concentration of the at least one biomarker is determined by enzyme linked-immunosorbent assay (ELISA), protein immunoprecipitation, immunoelectrophoresis, chemical analysis, SDS-PAGE and Western blot analysis, protein immunostaining, electrophoresis analysis, competitive binding assay, functional protein assay, protein microarray, high-performance liquid chromatography (HPLC), mass spectrometry, liquid chromatography-mass spectrometry (LC/MS), capillary electrophoresis (CE)-MS, cytometric bead array analysis, immunonephelometric assays, fluorescence activated cell sorting (FACS) analysis, microscopic analysis, microarray, or any combination thereof.
38. A kit for detecting and quantifying the level of one or more ALS biomarkers in a serum sample obtained from a patient having or suspected of having ALS, wherein the ALS biomarkers are selected from the group consisting of oxidized low-density lipoprotein receptor 1 (OLR1), oxidized low-density lipoprotein (ox-LDL), monocyte chemoattractant protein-1 MCP-1 (CCL2), interleukin-18 (IL-18), and any combination thereof.
39. The kit of embodiment 38, wherein the kit comprises a solid surface having an antibody or a fragment thereof comprising a binding epitope for the ALS biomarkers OLR1, ox-LDL, IL-17C, CCL2, IL-18, or any combination thereof, and at least one reagent for detecting formation of a biomarker-antibody complex.
40. The kit of embodiment 38 or embodiment 39 for use in selecting a patient for an ALS therapy.
41. The kit of any one of embodiments 38-40 for use in monitoring effectiveness of an ALS therapy, wherein effectiveness can be assessed during the course of or after completing an ALS therapy regimen.
42. A method of treating treatment-responsive ALS, comprising administering to a patient diagnosed with treatment-responsive ALS a therapeutically effective amount of an ALS therapy, wherein the treatment-responsive ALS is characterized as having at least one biomarker equal to or less than a reference concentration, wherein the at least one biomarker is selected from the group consisting of oxidized low-density lipoprotein receptor 1 (OLR1), oxidized low-density lipoprotein (ox-LDL), monocyte chemoattractant protein-1 MCP-1 (CCL2), and interleukin-18 (IL-18); and, wherein the reference concentration is an average concentration of the biomarker measured in serum samples collected from at least 10 healthy subjects having the same age, gender, and/or ethnicity as the patient.
43. The method of embodiment 42, wherein treatment-responsive ALS is characterized as having at least two biomarkers equal to or less than a reference concentration.
44. The method of embodiment 43, wherein the at least two or more biomarkers are CCL2 and IL-18.
45. The method of any one of embodiments 42-44, wherein the ALS therapy comprises Relyvrio (AMX0035), edaravone, riluzole, Treg infusion, IL-2, or any combination thereof.
46. The method of embodiment 45, wherein the ALS therapy is a Treg infusion, IL-2, or a combination thereof.
47. A method of determining whether a patient diagnosed with ALS is indicated as likely to be responsive to ALS treatment, comprising detecting the concentration of at least one biomarker compared to a reference concentration in a serum sample collected from the patient, wherein the at least one biomarker having a concentration equal to or less than the reference concentration indicates that the patient is likely to be responsive to ALS treatment, wherein the at least one biomarker is selected from the group consisting of oxidized low-density lipoprotein receptor 1 (OLR1), oxidized low-density lipoprotein (ox-LDL), monocyte chemoattractant protein-1 MCP-1 (CCL2), and interleukin-18 (IL-18); and, wherein the reference concentration is an average concentration of the biomarker measured in serum samples collected from at least 10 healthy subjects having the same age, gender, and/or ethnicity as the patient.
48. The method of embodiment 47, wherein at least two biomarkers having a concentration equal to or less than a reference concentration indicates that the patient is likely to be responsive to ALS treatment.
49. The method of embodiment 48, wherein the at least two or more biomarkers are CCL2 and IL-18.
50. The method of any one of embodiments 47-49, wherein the ALS therapy comprises Relyvrio (AMX0035), edaravone, riluzole, Treg infusion, IL-2, or any combination thereof.
51. The method of embodiment 50, wherein the ALS therapy is a Treg infusion, IL-2, or a combination thereof.

### 5. BRIEF DESCRIPTION OF THE FIGURES

The foregoing and other objects, features, and advantages will be apparent from the following description of particular embodiments of the invention, as illustrated in the accompanying drawings. The drawings are not necessarily to scale, emphasis instead being placed upon illustrating the principles of various embodiments of the invention.

FIGS. 1A-1L depicts graphs showing the assessment of the inflammatory analytes, CCL2 and IL-18, as biomarkers in progression and treatment of ALS. For the following graphs, *p<0.05, **p<0.01, ***p<0.001, and n.s.=not significant and the means and standard deviations vary among graphs due to the differences in ELISA plates used. FIG. 1A shows CCL2 levels in serum of ALS patients and healthy control (HC) subjects. FIG. 1B shows CCL2 levels in serum of rapidly progressing ALS patients, slowly progressing ALS patients, and HC subjects. FIG. 1C shows IL-18 levels in serum of ALS patients and HC subjects. FIG. 1D shows IL-18 levels in serum of rapidly progressing ALS patients, slowly progressing ALS patients, and HC subjects. FIGS. 1E-1G show correlation of CCL2 serum levels (FIG. 1E) and IL-18 serum levels (FIG. 1F) with ALS progression and with each other (FIG. 1G). FIGS. 1H-1J show CCL2 serum levels, IL-18 serum levels, and Appel ALS clinical scores (AALS) during a course of Treg/IL-2 combination therapy for subject 1 (FIG. 1H), subject 2 (FIG. 1I), and subject 3 (FIG. 1J) where arrows indicate Treg infusion dates, the dotted vertical lines demarcate Treg+IL-2 therapy or IL-2 therapy only intervals, the gray horizontal line is the mean value of each analyte in HC, and the black horizontal lines are the ± one standard deviation of each analyte level in HC. FIGS. 1K-1L show CCL2 serum levels (FIG. 1K) and IL-18 serum levels (FIG. 1L) for eight subjects during a course of Treg/IL-2 combination therapy where arrows demarcate the line corresponding to the individual patient, referred to by their subject number, the black horizontal line is the mean value of each analyte in HC, and the black dotted horizontal lines are the ± one standard deviation of each analyte level in HC.

FIGS. 2A-2L depicts graphs showing the assessment of the oxidative stress analytes, 4-HNE and MDA, as biomarkers in progression and treatment of ALS. For the following graphs, *p<0.05, **p<0.01, ***p<0.001, and n.s.=not significant and the means and standard deviations vary among graphs due to the differences in ELISA plates used. FIG. 2A shows 4-HNE levels in serum of ALS patients and HC subjects. FIG. 2B shows 4-HNE levels in serum of rapidly progressing ALS patients, slowly progressing ALS patients, and HC subjects. FIG. 2C shows MDA levels in serum of ALS patients and HC subjects. FIG. 2D shows MDA levels in serum of rapidly progressing ALS patients, slowly progressing ALS patients, and HC subjects. FIGS. 2E-2G show correlation of 4-HNE serum levels (FIG. 2E) and MDA serum levels (FIG. 2F) with ALS progression and with each other (FIG. 2G). FIGS. 2H-2J show 4-HNE serum levels, MDA serum levels, and Appel ALS clinical scores (AALS) during a course of Treg/IL-2 combination therapy for subject 1 (FIG. 2H), subject 2 (FIG. 2I), and subject 3 (FIG. 2J) where arrows indicate Treg infusion dates, the dotted vertical lines demarcate Treg+IL-2 therapy or IL-2 therapy only intervals, the gray horizontal line is the mean value of each analyte in HC, and the black horizontal lines are the ± one standard deviation of each analyte level in HC. FIGS. 2K-2L show 4-HNE serum levels (FIG. 2K) and MDA serum levels (FIG. 2L) for eight subjects during a course of Treg/IL-2 combination therapy where arrows demarcate the line corresponding to the individual patient, referred to by their subject number, the black horizontal line is the mean value of each analyte in HC, and the black dotted horizontal lines are the ± one standard deviation of each analyte level in HC.

FIGS. 3A-3L depicts graphs showing the assessment of the neuronal structural analytes, Nf-L and pNf-H, as biomarkers in progression and treatment of ALS. For the following graphs, *p<0.05, **p<0.01, ***p<0.001, and n.s.=not significant and the means and standard deviations vary among graphs due to the differences in ELISA plates used. FIG. 3A shows Nf-L levels in serum of ALS patients and HC subjects. FIG. 3B shows Nf-L levels in serum of rapidly progressing ALS patients, slowly progressing ALS patients, and HC subjects. FIG. 3C shows pNf-H levels in serum of ALS patients and HC subjects. FIG. 3D shows pNf-H levels in serum of rapidly progressing ALS patients, slowly progressing ALS patients, and HC subjects. FIGS. 3E-3G show correlation of Nf-L serum levels (FIG. 3E) and pNf-H serum levels (FIG. 3F) with ALS progression and with each other (FIG. 3G). FIGS. 3H-3J show Nf-L serum levels, pNf-H serum levels, and Appel ALS clinical scores (AALS), for subject 1 (FIG. 3H), subject 2 (FIG. 3I), and subject 3 (FIG. 3J) during a course of Treg/IL-2 combination therapy where arrows indicate Treg infusion dates, the dotted vertical lines demarcate Treg+IL-2 therapy or IL-2 therapy only intervals, the gray horizontal line is the mean value of each analyte in HC, and the black horizontal lines are the ± one standard deviation of each analyte level in HC. FIGS. 3K-3L show Nf-L serum levels (FIG. 3K) and pNf-H serum levels (FIG. 3L) for eight subjects during a course of Treg/IL-2 combination therapy where arrows demarcate the line corresponding to the individual patient, referred to by their subject number, the black horizontal line is the mean value of each analyte in HC, and the black dotted horizontal lines are the ± one standard deviation of each analyte level in HC.

### 6. DETAILED DESCRIPTION

In certain aspects, the present disclosure provides improved methods for monitoring amyotrophic lateral sclerosis (ALS) disease progression in a subject diagnosed as having or suspected of having ALS. Methods described herein comprise characterizing ALS and/or ALS disease progression in a subject by monitoring at least one biomarker in biological samples (e.g., blood, serum, PBMCs, CSF) collected from the subject. In certain embodiments, a biomarker can comprise a substance whose detection indicates a particular disease state (e.g., slowly progressing ALS, rapidly progressing ALS). More specifically, a biomarker may indicate a change in expression or state of a protein that correlates with the risk or progression of a disease, or with the susceptibility of the disease to a particular treatment.

Biomarkers of the present disclosure for methods of characterizing ALS and/or ALS disease progression in a subject are described in Section 6.1. The methods provided herein for selecting patients, determining the outcome of these methods, and/or serving as criteria in any way for these methods are described in Section 6.6 below. Methods of administering one or more ALS therapies and/or adjusting the administering of one or more ALS therapies are described in Section 6.5. Therefore, a person skilled in the art would understand that the methods provided herein include all permutations, including modifications, and combinations of the biomarkers disclosed herein, patients, dosing regimens, diagnostic and ALS staging criteria, and therapeutic outcomes as described above and below.

As used in this disclosure including the claims, the singular forms “a,” “an” and “the” include plural referents unless the context clearly dictates otherwise. The terms “a” (or “an”), as well as the terms “one or more,” and “at least one” can be used interchangeably herein unless the context clearly dictates otherwise.

The term “about” means within 20%, within 15%, within 10%, within 9%, within 8%, within 7%, within 6%, within 5%, within 4%, within 3%, within 2%, within 1%, or less variation of a given value or range. As used herein, a phrase such as “about A,” includes and is deemed to also recite “A.” As used herein, a phrase such as “about A to about B” includes and is deemed to also recite “A to B.”

The term “between” as used in a phrase as such “between A and B” or “between A-B” refers to a range including both A and B.

### 6.1 Biomarkers, Biomarker Detection, and Quantification Thereof

In certain embodiments, methods described herein involve a subject diagnosed as having or suspected of having ALS and can comprise characterizing ALS and/or ALS disease progression in a subject by monitoring at least one biomarker in biological sample collected from the subject. “Subjects” and “patients” are used synonymously herein and can refer to one or more of the patient populations described in Section 6.4.

As used herein, “biomarker” refers to a defined characteristic that is measured as an indicator of normal biological processes, pathogenic processes (e.g., progressive loss of motor neurons associated with ALS), or responses to an exposure or intervention, including therapeutic interventions. One or more specific substances in a biological sample of a patient, or the ratio of the level of one substance against another in the biological sample the aggregate level of one kind of substance (e.g., total protein, gene expression) in the biological sample, or the ratio involving such aggregate level, can be used as biomarkers. Non-limiting examples of biological samples include cerebrospinal fluid (CSF), serum, blood, peripheral blood mononuclear cells (PBMCs), urine, salvia, tissue, and the like. A biological sample can be collected from a patient using standard techniques known in the art. In particular embodiments, biological samples for use herein are serum samples.

In certain embodiments, the substance that serves as the one or more biomarkers in the methods disclosed herein can be any protein that reflects the presence and/or progression of ALS. In certain embodiments, a protein biomarker disclosed herein can be detected and quantified in a biological sample (e.g., serum) using any suitable method for protein detection and/or quantification known in the art. Such methods include, but are not limited to, immunoassays (e.g., enzyme linked-immunosorbent assay (ELISA)), protein immunoprecipitation, immunoelectrophoresis, chemical analysis, SDS-PAGE and Western blot analysis, protein immunostaining, electrophoresis analysis, competitive binding assays, functional protein assays, protein microarray, chromatography or spectrometry methods (e.g., high-performance liquid chromatography (HPLC), mass spectrometry, liquid chromatography-mass spectrometry (LC/MS), capillary electrophoresis (CE)-MS, or any separating front end coupled with MS detection and quantification), cytometric bead array analysis, immunonephelometric assays, fluorescence activated cell sorting (FACS) analysis, microscopic analysis, microarray, and the like. In some embodiments a protein biomarker, e.g., a protein biomarker present in a biological sample at a low level, may be detected using ultrasensitive methodologies and devices specifically designed for detecting low abundant proteins in a sample. Examples of such methodologies and devices include but are not limited to microfluidic analytical systems (see, e.g., Martel J M and Toner M., Annu Rev Biomed Eng (2014) July 11; 16:371-96); ultra-sensitive ELISA assays (see, e.g., Schubert S et al., Sci Rep (2015) 5, 11034); and nanoparticle-based systems (see, e.g., Li F et al., Biosensors and Bioelectronics (2015) 68, 626-632). One of skill in the art would appreciate that the method of measuring the biomarker(s) of the present disclosure in a biological sample may be chosen on the basis of the type of sample and/or the biomarker of interest. In specific embodiments, a biomarker disclosed herein can be detected and quantified in a biological sample (e.g., serum) using an ELISA. Kits for use in detecting and quantifying biomarkers disclosed herein are further described in Section 6.2.

In certain embodiments, a biomarker provided by the present disclosure is oxidized low density lipoprotein receptor 1 (OLR1; see, e.g., UniProtKB/Swiss-Prot Accession P78380 for an exemplary OLR1 sequence). OLR1 is also known in the art as lectin-type oxidized LDL receptor 1, LOX-1, LOXIN, SLOX1, scavenger receptor class E member 1, SCARE1, among others.

In certain embodiments, a biomarker provided by the present disclosure is oxidized low-density lipoprotein (ox-LDL). ox-LDL measures protein damage due to oxidative modification of the apolipoprotein B (ApoB) subunit on low density lipoprotein cholesterol (LDL-C). Thus, without being bound to any particular theory, ox-LDL measurements reflect lipid peroxidation and are markers of oxidant stress.

In certain embodiments, a biomarker provided by the present disclosure is soluble CD14 (sCD14; see, e.g., UniProtKB/Swiss-Prot Accession P08571 CD14_HUMAN for an exemplary sCD14 sequence). sCD14 is an acute phase protein, a class of proteins whose concentration in blood increases in response to inflammation.

In certain embodiments, a biomarker provided by the present disclosure is lipopolysaccharide binding protein (LBP; for an exemplary LBP sequence see, e.g., UniProtKB Accession P18428 LBP_Human). LBP is a soluble acute-phase protein that, without being bound to any particular theory, elicits immune responses.

In certain embodiments, a biomarker provided by the present disclosure is C Reactive Protein (CRP; for an exemplary CRP sequence see, e.g., UniProtKB Accession P02741 CRP_HUMAN). CRP is a protein made by the liver and, without being bound by any particular theory, the levels of CRP can increase when there is inflammation in the body of a subject.

In certain embodiments, a biomarker provided by the present disclosure is 4-hydroxynonenal (4-HNE; CAS Reg. No. 75899-68-2). A number of immunoassays to measure 4-HNE levels in serum are commercially available. In addition, since 4-HNE displays a relatively fast half-life of less than 2 minutes in normal physiological conditions, assays can detect levels of protein adducts of 4-HNE as a surrogate for 4-HNE. ELISA kits are commercially available for protein adducts of 4-HNE. An assay for determining 4-HNE adduct levels in serum is described, for instance, in Monroe et al., Bio Protoc (2019) 9(19): e3383.

In certain embodiments, a biomarker provided by the present disclosure is interleukin 6 (IL-6; for an exemplary IL-6 sequence see, e.g., UniProtKB Accession P05231 IL6_HUMAN). IL-6 is an interleukin that acts as both a pro-inflammatory cytokine and an anti-inflammatory myokine.

In certain embodiments, a biomarker provided by the present disclosure is interleukin 17F (IL-17F; for an exemplary IL-17F sequence see, e.g., UniProtKB Accession Q96PD4 IL17F_HUMAN). IL-17F is signaling protein and is considered a pro-inflammatory cytokine. In certain embodiments, a biomarker provided by the present disclosure is interleukin 17C (IL-17C; for an exemplary IL-17C sequence see, e.g., UniProtKB Accession Q9POM4 IL17C_HUMAN). IL-17C is an autocrine cytokine that can regulate innate epithelial immune responses.

In certain embodiments, a biomarker provided by the present disclosure is 4-malondialdehyde (MDA; CAS Reg. No. 542-78-9). Like 4-HNE, MDA is a major end-product of oxidation of polyunsaturated fatty acids and, without being bound by any particular theory, is frequently measured as an indicator of lipid peroxidation and oxidative stress.

In certain embodiments, a biomarker provided by the present disclosure is monocyte chemoattractant protein-1 (CCL2; for an exemplary CCL2 sequence see, e.g., UniProtKB Accession P13500 CCL2_HUMAN). CCL2 is a chemokine that can regulate migration and infiltration of monocytes and/or macrophages.

In certain embodiments, a biomarker provided by the present disclosure is interleukin-18 (IL-18; for an exemplary IL-18 sequence see, e.g., UniProtKB Accession Q14116 IL18_HUMAN). IL-18, also known as interferon-gamma inducing factor, is a proinflammatory cytokine.

In certain embodiments, a biomarker provided by the present disclosure is neurofilament light chain (Nf-L; for an exemplary Nf-L sequence see, e.g., UniProtKB Accession P07196 NFL_HUMAN). In certain embodiments, a biomarker provided by the present disclosure is phosphorylated neurofilament heavy subunit (pNf-H; for an exemplary Nf-H sequence see, e.g., UniProtKB Accession P12036 NFH_HUMAN). Without being bound to any particular theory, both Nf-L and pNf-H are believed to be markers of neuronal cell damage.

Any suitable combination of biomarkers described herein may be detected and quantified in accordance with the methods presented herein. Suitable biomarkers include, but are not limited to, proteins that are expressed in motor neurons. In certain embodiments, biomarkers for use in the methods disclosed herein can include at least one of the following biomarkers: OLR1, ox-LDL, sCD14, LBP, CRP, 4-HNE, IL-6, IL-17F, IL-17C, MDA, CCL2, IL-18, or any combination thereof. In certain embodiments, biomarkers for use in the methods disclosed herein can include at least one of the following biomarkers: ox-LDL, 4-HNE, MDA, CCL2, IL-18, or any combination thereof. In certain embodiments, biomarkers for use in the methods disclosed herein can include at least one of the following biomarkers: ox-LDL, 4-HNE, CCL2, IL-18, or any combination thereof. In certain embodiments, biomarkers for use in the methods disclosed herein can include at least one of the following biomarkers: 4-HNE, MDA, CCL2, IL-18, or any combination thereof. In certain embodiments, biomarkers for use in the methods disclosed herein can include at least one of the following biomarkers: 4-HNE, CCL2, IL-18, or any combination thereof. In certain embodiments, biomarkers for use in the methods disclosed herein can comprise one or more inflammatory analytes (e.g., CCL2, IL-18). In certain embodiments, biomarkers for use in the methods disclosed herein can comprise one or more oxidative stress analytes (e.g., 4-HNE, MDA, OLR1, ox-LDL). In certain embodiments, biomarkers for use in the methods disclosed herein can include at least one of the following biomarkers: OLR1, ox-LDL, Nf-L, pNf-H, sCD14, LBP, CRP, 4-HNE, IL-6, IL-17F, IL-17C, MDA, CCL2, IL-18, or any combination thereof.

In certain embodiments of the methods provided herein, the at least one biomarker comprises OLR1, ox-LDL, Nf-L, pNf-H, sCD14, LBP, CRP, 4-HNE, IL-6, IL-17F, IL-17C, MDA, CCL2, or IL-18. In some embodiments of the methods provided herein, the at least one biomarker comprises OLR1, ox-LDL, sCD14, LBP, CRP, 4-HNE, IL-6, IL-17F, IL-17C, MDA, CCL2, or IL-18. In some embodiments of the methods provided herein, the at least one biomarker comprises OLR1, ox-LDL, sCD14, LBP, CRP, 4-HNE, IL-17F, IL-17C, MDA, CCL2, or IL-18. In some embodiments of the methods provided herein, the at least one biomarker comprises OLR1, ox-LDL, sCD14, LBP, CRP, 4-HNE, IL-17F, IL-17C, CCL2, or IL-18. In some embodiments of the methods provided herein, the at least one biomarker comprises ox-LDL, 4-HNE, CCL2, MDA or IL-18. In some embodiments of the methods provided herein, the at least one biomarker comprises ox-LDL, 4-HNE, CCL2, or IL-18. In some embodiments of the methods provided herein, the at least one biomarker comprises 4-HNE, CCL2, MDA or IL-18. In some embodiments of the methods provided herein, the at least one biomarker comprises 4-HNE, CCL2, or IL-18. In some embodiments of the methods provided herein, the at least one biomarker comprises OLR1, ox-LDL, 4-HNE, CCL2, or IL-18. In some embodiments of the methods provided herein, the at least one biomarker comprises OLR1, ox-LDL, or 4-HNE. In some embodiments of the methods provided herein, the at least one biomarker comprises OLR1 or ox-LDL. In some embodiments of the methods provided herein, the at least one biomarker comprises CCL2 or IL-18. In some embodiments of the methods provided herein, the at least one biomarker comprises OLR1, ox-LDL, CCL2 or IL-18.

In certain embodiments, any suitable number of biomarkers can be detected and quantified. In some embodiments, methods disclosed herein can comprise detecting and quantifying up to about 1 biomarker, up to about 2 biomarkers, up to about 3 biomarkers, up to about 4 biomarkers, up to about 5 biomarkers, up to about 6 biomarkers, up to about 7 biomarkers, up to about 8 biomarkers, up to about 9 biomarkers, up to about 10 biomarkers, up to about 11 biomarkers, up to about 12 biomarkers, up to about 13 biomarkers, up to about 14 biomarkers, up to about 15 biomarkers, up to about 20 biomarkers, or more than 20 biomarkers wherein the biomarkers can be any of those disclosed herein alone or in combination with one or more biomarkers for detecting ALS.

In some embodiments, methods disclosed herein can comprise detecting and quantifying up to about 1 biomarker, up to about 2 biomarkers, up to about 3 biomarkers, up to about 4 biomarkers, up to about 5 biomarkers, up to about 6 biomarkers, up to about 7 biomarkers, up to about 8 biomarkers, up to about 9 biomarkers, up to about 10 biomarkers, up to about 11 biomarkers, up to about 12 biomarkers, up to about 13 biomarkers, or up to about 14 biomarkers, wherein the biomarkers comprise OLR1, ox-LDL, Nf-L, pNf-H, sCD14, LBP, CRP, 4-HNE, IL-6, IL-17F, IL-17C, MDA, CCL2, and/or IL-18. In some embodiments, methods disclosed herein comprise detecting and quantifying at least about 1 biomarker, at least about 2 biomarkers, at least about 3 biomarkers, at least about 4 biomarkers, at least about 5 biomarkers, at least about 6 biomarkers, at least about 7 biomarkers, at least about 8 biomarkers, at least about 9 biomarkers, at least about 10 biomarkers, at least about 11 biomarkers, at least about 12 biomarkers, at least about 13 biomarkers, or at least about 14 biomarkers, wherein the biomarkers comprise OLR1, ox-LDL, Nf-L, pNf-H, sCD14, LBP, CRP, 4-HNE, IL-6, IL-17F, IL-17C, MDA, CCL2, and/or IL-18. In some embodiments, methods disclosed herein comprise detecting and quantifying between about 1 to about 14 biomarkers, between about 1 to about 13 biomarkers, between about 1 to about 12 biomarkers, between about 1 to about 11 biomarkers, between about 1 to about 10 biomarkers, between about 1 to about 9 biomarkers, between about 1 to about 8 biomarkers, between about 1 to about 7 biomarkers, between about 1 to about 6 biomarkers, between about 1 to about 5 biomarkers, between about 1 to about 4 biomarkers, between about 1 to about 3 biomarkers, or between about 1 to about 2 biomarkers, wherein the biomarkers comprise OLR1, ox-LDL, Nf-L, pNf-H, sCD14, LBP, CRP, 4-HNE, IL-6, IL-17F, IL-17C, MDA, CCL2, and/or IL-18. In some embodiments, methods disclosed herein comprise detecting and quantifying about 1 biomarker, about 2 biomarkers, about 3 biomarkers, about 4 biomarkers, about 5 biomarkers, about 6 biomarkers, about 7 biomarkers, about 8 biomarkers, about 9 biomarkers, about 10 biomarkers, about 11 biomarkers, about 12 biomarkers, about 13 biomarkers, or about 14 biomarkers, wherein the biomarkers comprise OLR1, ox-LDL, Nf-L, pNf-H, sCD14, LBP, CRP, 4-HNE, IL-6, IL-17F, IL-17C, MDA, CCL2, and/or IL-18.

In some embodiments, methods disclosed herein can comprise detecting and quantifying up to about 1 biomarker, up to about 2 biomarkers, up to about 3 biomarkers, up to about 4 biomarkers, up to about 5 biomarkers, up to about 6 biomarkers, up to about 7 biomarkers, up to about 8 biomarkers, up to about 9 biomarkers, up to about 10 biomarkers, up to about 11 biomarkers, or up to about 12 biomarkers, wherein the biomarkers comprise OLR 1, ox-LDL, sCD14, LBP, CRP, 4-HNE, IL-6, IL-17F, IL-17C, MDA, CCL2, and/or IL-18. In some embodiments, methods disclosed herein comprise detecting and quantifying at least about 1 biomarker, at least about 2 biomarkers, at least about 3 biomarkers, at least about 4 biomarkers, at least about 5 biomarkers, at least about 6 biomarkers, at least about 7 biomarkers, at least about 8 biomarkers, at least about 9 biomarkers, at least about 10 biomarkers, at least about 11 biomarkers, or at least about 12 biomarkers, wherein the biomarkers comprise OLR1, ox-LDL, sCD14, LBP, CRP, 4-HNE, IL-6, IL-17F, IL-17C, MDA, CCL2, and/or IL18. In some embodiments, methods disclosed herein comprise detecting and quantifying between about 1 to about 12 biomarkers, between about 1 to about 11 biomarkers, between about 1 to about 10 biomarkers, between about 1 to about 9 biomarkers, between about 1 to about 8 biomarkers, between about 1 to about 7 biomarkers, between about 1 to about 6 biomarkers, between about 1 to about 5 biomarkers, between about 1 to about 4 biomarkers, between about 1 to about 3biomarkers, or between about 1 to about 2 biomarkers, wherein the biomarkers comprise OLR1, ox-LDL, sCD14, LBP, CRP, 4-HNE, IL-6, IL-17F, IL-17C, MDA, CCL2, and/or IL18. In some embodiments, methods disclosed herein comprise detecting and quantifying about 1 biomarker, about 2 biomarkers, about 3 biomarkers, about 4 biomarkers, about 5 biomarkers, about 6 biomarkers, about 7 biomarkers, about 8 biomarkers, about 9 biomarkers, about 10 biomarkers, about 11 biomarkers, or about 12 biomarkers, wherein the biomarkers comprise OLR1, ox-LDL, sCD14, LBP, CRP, 4-HNE, IL-6, IL-17F, IL-17C, MDA, CCL2, and/or IL18.

In some embodiments, methods disclosed herein can comprise detecting and quantifying up to about 1 biomarker, up to about 2 biomarkers, up to about 3 biomarkers, up to about 4 biomarkers, up to about 5 biomarkers, up to about 6 biomarkers, up to about 7 biomarkers, up to about 8 biomarkers, up to about 9 biomarkers, up to about 10 biomarkers, or up to about 11 biomarkers, wherein the biomarkers comprise OLR 1, ox-LDL, sCD 14, LBP, CRP, 4-HNE, IL-17F, IL-17C, MDA, CCL2, and/or IL18. In some embodiments, methods disclosed herein comprise detecting and quantifying at least about 1 biomarker, at least about 2 biomarkers, at least about 3 biomarkers, at least about 4 biomarkers, at least about 5 biomarkers, at least about 6 biomarkers, at least about 7 biomarkers, at least about 8 biomarkers, at least about 9 biomarkers, at least about 10 biomarkers, or at least about 11 biomarkers, wherein the biomarkers comprise OLR1, ox-LDL, sCD14, LBP, CRP, 4-HNE, IL-17F, IL-17C, MDA, CCL2, and/or IL18. In some embodiments, methods disclosed herein comprise detecting and quantifying between 1 to about 11 biomarkers, between about 1 to about 10 biomarkers, between about 1 to about 9 biomarkers, between about 1 to about 8 biomarkers, between about 1 to about 7 biomarkers, between about 1 to about 6 biomarkers, between about 1 to about 5 biomarkers, between about 1 to about 4 biomarkers, between about 1 to about 3 biomarkers, or between about 1 to about 2 biomarkers, wherein the biomarkers comprise OLR1, ox-LDL, sCD14, LBP, CRP, 4-HNE, IL-17F, IL-17C, MDA, CCL2, and/or IL18. In some embodiments, methods disclosed herein comprise detecting and quantifying about 1 biomarker, about 2 biomarkers, about 3 biomarkers, about 4 biomarkers, about 5 biomarkers, about 6 biomarkers, about 7 biomarkers, about 8 biomarkers, about 9 biomarkers, about 10 biomarkers, or about 11 biomarkers, wherein the biomarkers comprise OLR1, ox-LDL, sCD14, LBP, CRP, 4-HNE, IL-17F, IL-17C, MDA, CCL2, and/or IL-18.

In some embodiments, methods disclosed herein can comprise detecting and quantifying up to about 1 biomarker, up to about 2 biomarkers, up to about 3 biomarkers, up to about 4 biomarkers, up to about 5 biomarkers, up to about 6 biomarkers, up to about 7 biomarkers, up to about 8 biomarkers, up to about 9 biomarkers, or up to about 10 biomarkers, wherein the biomarkers comprise OLR1, ox-LDL, sCD14, LBP, CRP, 4-HNE, IL-17F, IL-17C, CCL2, and/or IL-18. In some embodiments, methods disclosed herein comprise detecting and quantifying at least about 1 biomarker, at least about 2 biomarkers, at least about 3 biomarkers, at least about 4 biomarkers, at least about 5 biomarkers, at least about 6 biomarkers, at least about 7 biomarkers, at least about 8 biomarkers, at least about 9 biomarkers, or at least about 10 biomarkers, wherein the biomarkers comprise OLR1, ox-LDL, sCD14, LBP, CRP, 4-HNE, IL-17F, IL-17C, CCL2, and/or IL-18. In some embodiments, methods disclosed herein comprise detecting and quantifying between 1 to about 10 biomarkers, between about 1 to about 9 biomarkers, between about 1 to about 8 biomarkers, between about 1 to about 7 biomarkers, between about 1 to about 6 biomarkers, between about 1 to about 5 biomarkers, between about 1 to about 4 biomarkers, between about 1 to about 3 biomarkers, or between about 1 to about 2 biomarkers, wherein the biomarkers comprise OLR1, ox-LDL, sCD14, LBP, CRP, 4-HNE, IL-17F, IL-17C, CCL2, and/or IL-18. In some embodiments, methods disclosed herein comprise detecting and quantifying about 1 biomarker, about 2 biomarkers, about 3 biomarkers, about 4 biomarkers, about 5 biomarkers, about 6 biomarkers, about 7 biomarkers, about 8 biomarkers, about 9 biomarkers, or about 10 biomarkers, wherein the biomarkers comprise OLR1, ox-LDL, sCD14, LBP, CRP, 4-HNE, IL-17F, IL-17C, CCL2, and/or IL-18.

In some embodiments, methods disclosed herein can comprise detecting and quantifying up to about 1 biomarker, up to about 2 biomarkers, up to about 3 biomarkers, up to about 4 biomarkers, or up to about 5 biomarkers, wherein the biomarkers comprise OLR1, ox-LDL, 4-HNE, CCL2, and/or IL-18. In some embodiments, methods disclosed herein comprise detecting and quantifying at least about 1 biomarker, at least about 2 biomarkers, at least about 3 biomarkers, at least about 4 biomarkers, or at least about 5 biomarkers, wherein the biomarkers comprise OLR1, ox-LDL, 4-HNE, CCL2, and/or IL-18. In some embodiments, methods disclosed herein comprise detecting and quantifying between about 1 to about 5 biomarkers, between about 1 to about 4 biomarkers, between about 1 to about 3 biomarkers, or between about 1 to about 2 biomarkers, wherein the biomarkers comprise OLR1, ox-LDL, 4-HNE, CCL2, and/or IL-18. In some embodiments, methods disclosed herein comprise detecting and quantifying about 1 biomarker, about 2 biomarkers, about 3 biomarkers, about 4 biomarkers, or about 5 biomarkers, wherein the biomarkers comprise OLR1, ox-LDL, 4-HNE, CCL2, and/or IL-18.

In some embodiments, methods disclosed herein can comprise detecting and quantifying up to about 1 biomarker, up to about 2 biomarkers, up to about 3 biomarkers, up to about 4 biomarkers, or up to about 5 biomarkers, wherein the biomarkers comprise ox-LDL, 4-HNE, CCL2, MDA and/or IL18. In some embodiments, methods disclosed herein comprise detecting and quantifying at least about 1 biomarker, at least about 2 biomarkers, at least about 3 biomarkers, at least about 4 biomarkers, or at least about 5 biomarkers, wherein the biomarkers comprise ox-LDL, 4-HNE, CCL2, MDA and/or IL18. In some embodiments, methods disclosed herein comprise detecting and quantifying between about 1 to about 5 biomarkers, between about 1 to about 4 biomarkers, between about 1 to about 3 biomarkers, or between about 1 to about 2 biomarkers, wherein the biomarkers comprise ox-LDL, 4-HNE, CCL2, MDA and/or IL18. In some embodiments, methods disclosed herein comprise detecting and quantifying about 1 biomarker, about 2 biomarkers, about 3 biomarkers, about 4 biomarkers, or about 5 biomarkers, wherein the biomarkers comprise ox-LDL, 4-HNE, CCL2, MDA and/or IL18.

In some embodiments, methods disclosed herein can comprise detecting and quantifying up to about 1 biomarker, up to about 2 biomarkers, up to about 3 biomarkers, or up to about 4 biomarkers, wherein the biomarkers comprise ox-LDL, 4-HNE, CCL2, and or IL-18. In some embodiments, methods disclosed herein comprise detecting and quantifying at least about 1 biomarker, at least about 2 biomarkers, at least about 3 biomarkers, or at least about 4 biomarkers, wherein the biomarkers comprise ox-LDL, 4-HNE, CCL2, and/or IL-18. In some embodiments, methods disclosed herein comprise detecting and quantifying between about 1 to about 4 biomarkers, between about 1 to about 3 biomarkers, or between about 1 to about 2 biomarkers, wherein the biomarkers comprise ox-LDL, 4-HNE, CCL2, and/or IL-18. In some embodiments, methods disclosed herein comprise detecting and quantifying about 1 biomarker, about 2 biomarkers, about 3 biomarkers, or about 4 biomarkers, wherein the biomarkers comprise ox-LDL, 4-HNE, CCL2, and/or IL-18.

In some embodiments, methods disclosed herein can comprise detecting and quantifying up to about 1 biomarker, up to about 2 biomarkers, up to about 3 biomarkers, or up to about 4 biomarkers, wherein the biomarkers comprise OLR1, ox-LDL, CCL2, and/or IL-18. In some embodiments, methods disclosed herein comprise detecting and quantifying at least about 1 biomarker, at least about 2 biomarkers, at least about 3 biomarkers, or at least about 4 biomarkers, wherein the biomarkers comprise OLR1, ox-LDL, CCL2, and/or IL-18. In some embodiments, methods disclosed herein comprise detecting and quantifying between about 1 to about 4 biomarkers, between about 1 to about 3 biomarkers, or between about 1 to about 2 biomarkers, wherein the biomarkers comprise OLR1, ox-LDL, CCL2, and/or IL-18. In some embodiments, methods disclosed herein comprise detecting and quantifying about 1 biomarker, about 2 biomarkers, about 3 biomarkers, or about 4 biomarkers, wherein the biomarkers comprise OLR1, ox-LDL, CCL2, and/or IL-18.

In some embodiments, methods disclosed herein can comprise detecting and quantifying up to about 1 biomarker, up to about 2 biomarkers, up to about 3 biomarkers, or up to about 4 biomarkers, wherein the biomarkers comprise 4-HNE, CCL2, MDA and/or IL18. In some embodiments, methods disclosed herein comprise detecting and quantifying at least about 1 biomarker, at least about 2 biomarkers, at least about 3 biomarkers, or at least about 4 biomarkers, wherein the biomarkers comprise 4-HNE, CCL2, MDA and/or IL18. In some embodiments, methods disclosed herein comprise detecting and quantifying between about 1 to about 4 biomarkers, between about 1 to about 3 biomarkers, or between about 1 to about 2 biomarkers, wherein the biomarkers comprise 4-HNE, CCL2, MDA and/or IL18. In some embodiments, methods disclosed herein comprise detecting and quantifying about 1 biomarker, about 2 biomarkers, about 3 biomarkers, or about 4 biomarkers, wherein the biomarkers comprise 4-HNE, CCL2, MDA and/or IL18.

In some embodiments, methods disclosed herein can comprise detecting and quantifying up to about 1 biomarker, up to about 2 biomarkers, or up to about 3 biomarkers, wherein the biomarkers comprise OLR1, ox-LDL, and/or 4-HNE. In some embodiments, methods disclosed herein comprise detecting and quantifying at least about 1 biomarker, at least about 2 biomarkers, or at least about 3 biomarkers, wherein the biomarkers comprise OLR1, ox-LDL, and/or 4-HNE. In some embodiments, methods disclosed herein comprise detecting and quantifying between about 1 to about 3 biomarkers, or between about 1 to about 2 biomarkers, wherein the biomarkers comprise OLR1, ox-LDL, and/or 4-HNE. In some embodiments, methods disclosed herein comprise detecting and quantifying about 1 biomarker, about 2 biomarkers, or about 3 biomarkers, wherein the biomarkers comprise OLR1, ox-LDL, and/or 4-HNE.

In some embodiments, methods disclosed herein can comprise detecting and quantifying up to about 1 biomarker, up to about 2 biomarkers, or up to about 3 biomarkers, wherein the biomarkers comprise 4-HNE, CCL2, and/or IL-18. In some embodiments, methods disclosed herein comprise detecting and quantifying at least about 1 biomarker, at least about 2 biomarkers, or at least about 3 biomarkers, wherein the biomarkers comprise 4-HNE, CCL2, and/or IL-18. In some embodiments, methods disclosed herein comprise detecting and quantifying between about 1 to about 3 biomarkers, or between about 1 to about 2 biomarkers, wherein the biomarkers comprise 4-HNE, CCL2, and/or IL-18. In some embodiments, methods disclosed herein comprise detecting and quantifying about 1 biomarker, about 2 biomarkers, or about 3 biomarkers, wherein the biomarkers comprise 4-HNE, CCL2, and/or IL-18.

In some embodiments, methods disclosed herein can comprise detecting and quantifying up to about 1 biomarker or up to about 2 biomarkers, wherein the biomarkers comprise OLR1 and/or ox-LDL. In some embodiments, methods disclosed herein comprise detecting and quantifying at least about 1 biomarker or at least about 2 biomarkers, wherein the biomarkers comprise OLR1 and/or ox-LDL. In some embodiments, methods disclosed herein comprise detecting and quantifying between about 1 to about 2 biomarkers, wherein the biomarkers comprise OLR1 and/or ox-LDL. In some embodiments, methods disclosed herein comprise detecting and quantifying about 1 biomarker or about 2 biomarkers, wherein the biomarkers comprise OLR1 and/or ox-LDL.

In some embodiments, methods disclosed herein can comprise detecting and quantifying up to about 1 biomarker or up to about 2 biomarkers, wherein the biomarkers comprise CCL2 and/or IL-18. In some embodiments, methods disclosed herein comprise detecting and quantifying at least about 1 biomarker or at least about 2 biomarkers, wherein the biomarkers comprise CCL2 and/or IL-18. In some embodiments, methods disclosed herein comprise detecting and quantifying between about 1 to about 2 biomarkers, wherein the biomarkers comprise CCL2 and/or IL-18. In some embodiments, methods disclosed herein comprise detecting and quantifying about 1 biomarker or about 2 biomarkers, wherein the biomarkers comprise CCL2 and/or IL-18.

In certain embodiments of the methods provided herein, a concentration of a biomarker can be determined and/or quantified by comparing to a baseline concentration. A baseline concentration, or “baseline”, for determination and/or quantification of at least one biomarker can comprise measuring the concentration of the least one biomarker in a biological sample collected from the patient before administering an ALS therapy.

In some embodiments, a baseline concentration of a biomarker is measured in a biological sample collected from a patient before administering an ALS therapy. In some embodiments, a baseline concentration of a biomarker is measured in a biological sample collected from a patient naïve to ALS therapy before administering an ALS therapy. In some embodiments, a baseline concentration of a biomarker is measured in a biological sample collected from a patient at least about one week, at least about 5 days, at least about 3 days, or at least about 1 day before administering an ALS therapy. In some embodiments, a baseline concentration of a biomarker is measured in a biological sample collected from a patient less than one day before administering an ALS therapy, e.g., about 12 hours, about 8 hours, about 4 hours, about 1 hour, about 30 minutes or about less than 30 minutes before administering an ALS therapy.

In certain embodiments, a baseline concentration of a biomarker is measured in a biological sample collected from a patient before administering an ALS therapy wherein the patient has previously received at least one ALS therapy, at least two ALS therapies, at least three ALS therapies, or more than about three ALS therapies. In some embodiments, a baseline concentration of a biomarker is measured in a biological sample collected from a patient at least about one month, at least about three weeks, at least about two weeks, at least about one week, at least about 5 days, at least about 3 days, or at least about 1 day after completing a first ALS therapy and before administering a second ALS therapy. In some embodiments, a baseline concentration of a biomarker is measured in a biological sample collected from a patient at least about one month, at least about three weeks, at least about two weeks, about one week, at least about 5 days, at least about 3 days, or at least about 1 day after completing a first ALS therapy and at least about one week, at least about 5 days, at least about 3 days, or at least about 1 day before administering an ALS therapy. In some embodiments, a baseline concentration of a biomarker is measured in a biological sample collected from a patient after completing one dosage regimen of ALS therapy and before administering a subsequent dosage regimen of ALS therapy to the patient.

In some embodiments, a baseline concentration of a biomarker is measured in a serum sample collected from a patient before administering an ALS therapy. In some embodiments, a baseline concentration of a biomarker is measured in a serum sample collected from a patient naïve to ALS therapy before administering an ALS therapy. In some embodiments, a baseline concentration of a biomarker is measured in a serum sample collected from a patient at least about one week, at least about 5 days, at least about 3 days, or at least about 1 day before administering an ALS therapy. In some embodiments, a baseline concentration of a biomarker is measured in a serum sample collected from a patient less than one day before administering an ALS therapy, e.g., about 12 hours, about 8 hours, about 4 hours, about 1 hour, about 30 minutes or about less than 30 minutes before administering an ALS therapy.

In certain embodiments, a baseline concentration of a biomarker is measured in a serum sample collected from a patient before administering an ALS therapy wherein the patient has previously received at least one ALS therapy, at least two ALS therapies, at least three ALS therapies, or more than about three ALS therapies. In some embodiments, a baseline concentration of a biomarker is measured in a serum sample collected from a patient at least about one month, at least about three weeks, at least about two weeks, at least about one week, at least about 5 days, at least about 3 days, or at least about 1 day after completing a first ALS therapy and before administering a second ALS therapy. In some embodiments, a baseline concentration of a biomarker is measured in a serum sample collected from a patient at least about one month, at least about three weeks, at least about two weeks, about one week, at least about 5 days, at least about 3 days, or at least about 1 day after completing a first ALS therapy and at least about one week, at least about 5 days, at least about 3 days, or at least about 1 day before administering an ALS therapy. In some embodiments, a baseline concentration of a biomarker is measured in a serum sample collected from a patient after completing one dosage regimen of ALS therapy and before administering a subsequent dosage regimen of ALS therapy to the patient.

In certain embodiments of the methods provided herein, a concentration of a biomarker can be determined and/or quantified by comparing to a reference concentration. In certain embodiments of the methods provided, a concentration of a biomarker measured in serum samples can be determined and/or quantified by comparing to a reference concentration measured in serum samples. In some embodiments, for example, a concentration of a biomarker can be determined to be elevated if the concentration of the biomarker in a sample (e.g., serum), when compared to a reference concentration, is higher than a reference concentration. In some other embodiments, for example, a concentration of a biomarker can be determined to be lowered if the concentration of the biomarker in a sample (e.g., serum), when compared to a reference concentration, is less than a reference concentration. One of skill in the art would understand that a given biomarker concentration is compared to a reference concentration specific to that biomarker. For example, an ox-LDL concentration is compared to a reference concentration for ox-LDL whereas an IL-18 concentration is compared to a reference concentration for IL-18, and so forth for any given biomarker.

In certain embodiments, a reference concentration is obtained from one or more healthy individuals. In certain embodiments, a reference concentration is a mean average concentration from healthy individuals. In some embodiments, a reference concentration is one standard deviation above the mean average concentration from a group of healthy individuals. In some embodiments, a reference concentration is a range of concentrations encompassing a mean average concentration from a group of healthy individuals.

The number of healthy individuals that contribute to a reference concentration can, for example, be at least 3. In some embodiments, the number of healthy individuals that contribute to a reference concentration can, for example, be at least 10. In certain embodiments, the number of healthy individuals is between 3 to about 10,000. In other embodiments, the number of healthy individuals is between 5 to about 1,000, or between 8 to 40. In some embodiments, the number healthy individuals contributing to the reference concentration is at least 3, at least 4, at least 5, at least 6, at least 7, at least 8, at least 9, at least 10, at least 15, at least 20, at least 25, at least 30, at least 35, at least 40, at least 50, at least 75, at least 100, at least 150, at least 200, at least 250, at least 300, at least 400, at least 500, at least 750, at least 1,000, at least 2,000, or at least 3,000.

In certain embodiments, the reference concentration is from healthy individuals that are age-matched to the ALS patient. In certain embodiments, the reference concentration is from healthy individuals that are gender-matched to the ALS patient. In certain embodiments, the reference concentration is from healthy individuals that share the same ethnicity as the ALS patient. In certain embodiments, the reference concentration is an average concentration of the biomarker measured in serum samples collected from at least 3, at least 4, at least 5, at least 6, at least 7, at least 8, at least 9, at least 10, or more than 10 healthy subjects having the same age, gender, and/or ethnicity as the patient.

In some embodiments, a reference concentration for sCD 14 can be from about 1.9 μg/mL to about 3.1 μg/mL. from about 2 μg/mL to about 2.9 μg/mL, or from about 2.1 μg/mL to about 2.7 μg/mL. In some embodiments, a reference concentration for LBP can be from about 5 μg/mL to about 30 μg/mL, or from about 10 μg/mL to about 25 μg/mL. In some embodiments, a reference concentration for IL-6 can be from about 0.8 pg/mL to about 4.1 pg/mL, from about 2.8 pg/mL to about 4 pg/mL, or from about 3.2 pg/mL to about 3.7 pg/mL. In some embodiments, a reference concentration for IL-17F can be from below a level of detection to about 4.5 pg/mL, from below a level of detection to about 1 pg/mL, from about 0.01 pg/mL to about 2.0 pg/mL, or from about 0.01 pg/mL to about 1.0 pg/mL. In some embodiments, a reference concentration for IL-17C can be from about 4 pg/mL to about 19 pg/mL, from about 5 pg/mL to about 14 pg/mL, from about 5 pg/mL to about 11 pg/mL, or from about 7 pg/mL to about 15 pg/mL. In some embodiments, a reference concentration for CRP can be from about 0.3 μg/mL to about 2.2 μg/mL, from about 0.5 μg/mL to about 2.0 μg/mL, or from about 1 μg/mL to about 2.0 μg/mL. In some embodiments, a reference concentration for 4-HNE can be from about 0.5 μg/mL to about 12 μg/mL, from about 1 μg/mL to about 10 μg/mL, or from about 2 μg/mL to about 9 μg/mL. In some embodiments, a reference concentration for ox-LDL can be from about 48 U/L to about 100 U/L, from about 50 U/L to about 70 U/L, or from about 70 U/L to about 90 U/L. In some embodiments, a reference concentration for OLR 1 can be from about 150 pg/mL to about 450 pg/mL, from about 160 pg/mL to about 425 pg/mL, or from about 250 pg/mL to about 400 pg/mL. In some embodiments, a reference concentration for MDA can be from about 1000 ng/mL to about 700 ng/mL, from about 950 ng/ml to about 750 ng/ml, or from about 900 ng/mL to about 800 ng/mL. In some embodiments, a reference concentration for CCL2 can be from about 500 pg/mL to about 100 pg/mL, from about 400 pg/mL to about 200 pg/mL, or from about 350 pg/mL to about 150 pg/mL. In some embodiments, a reference concentration for IL-18 can be from about 300 pg/mL to about 10 pg/mL, from about 200 pg/mL to about 50 pg/mL, or from about 150 pg/mL to about 75 pg/mL.

### 6.2 Kits

Also provided in the present disclosure are kits for determining the level of one or more of the biomarkers disclosed herein. In certain embodiments, the kits described herein can be used to assess the rate of progression of ALS in a subject and/or to determine the likelihood of a subject with ALS having slow or rapid progressing ALS. In some embodiments, kits disclosed herein can be used in selecting a patient for an ALS therapy. In certain embodiments, kits disclosed herein can be used in monitoring the effectiveness of an ALS therapy, wherein effectiveness can be assessed during the course of or after completing an ALS therapy regimen. In certain embodiments, kits disclosed herein can be used in determining whether ALS therapy should be stopped, the dosage of the ALS therapy should be changed, a new ALS therapy should be administered instead, and/or an additional ALS therapy should be administered concurrently with the therapy currently being administered to the subject.

In certain embodiments, kits described herein can be used for determining the level of a biomarker in a subject, wherein the kit comprises an antigen-binding molecule specific for the biomarker which allows for measuring the level of the biomarker in a biological sample In certain embodiments, a kit comprises an antigen-binding molecule specific for OLR1, ox-LDL, Nf-L, pNf-H, sCD14, LBP, CRP, 4-HNE, IL-6, IL-17F, IL-17C, MDA, CCL2, IL-18, or any combination thereof. In certain embodiments, a kit comprises an antigen-binding molecule specific for OLR1, ox-LDL, sCD14, LBP, CRP, 4-HNE, IL-6, IL-17F, IL-17C, MDA, CCL2, and/or IL18. In certain embodiments, a kit comprises an antigen-binding molecule specific for OLR1, ox-LDL, sCD14, LBP, CRP, 4-HNE, IL-17F, IL-17C, MDA, CCL2, and/or IL18. In certain embodiments, a kit comprises an antigen-binding molecule specific for OLR1, ox-LDL, sCD14, LBP, CRP, 4-HNE, IL-17F, IL-17C, CCL2, and/or IL-18. In certain embodiments, a kit comprises an antigen-binding molecule specific for OLR1, ox-LDL, 4-HNE, CCL2, and/or IL-18. In certain embodiments, a kit comprises an antigen-binding molecule specific for OLR1, ox-LDL, CCL2, and/or IL-18. In certain embodiments, a kit comprises an antigen-binding molecule specific for OLR1, ox-LDL, and/or 4-HNE. In certain embodiments, a kit comprises an antigen-binding molecule specific for CCL2 and/or IL-18. In certain embodiments, a kit comprises an antigen-binding molecule specific for OLR1, ox-LDL, CCL2 and IL-18. In certain embodiments, a kit comprises an antigen-binding molecule specific for ox-LDL, 4-HNE, MDA, CCL2, and/or IL18. In certain embodiments, a kit comprises an antigen-binding molecule specific for ox-LDL, 4-HNE, CCL2, and/or IL-18. In certain embodiments, a kit comprises an antigen-binding molecule specific for 4-HNE, MDA, CCL2, and/or IL18. In certain embodiments, a kit comprises an antigen-binding molecule specific for 4-HNE, CCL2, an/or IL-18.

In certain embodiments, the kits disclosed herein can also comprise suitable detection agents, including for example, conjugates and substrates to facilitate detection (e.g., antibodies labelled with streptavidin, biotin, horseradish peroxidase, etc.). In certain embodiments, kits disclosed herein comprise at least one reagent for detecting formation of a biomarker-antibody complex. In certain embodiments, kits disclosed herein can also feature various devices and additional reagents and/or buffers, and/or printed instructional materials for using the kit to detect and/or quantify levels of one or more biomarkers. The kit reagents described herein, which may be optionally associated with detectable labels, can be presented in the format of a plurality of beads, a multiwall plate, a microarray, slide, microfluidics card, or a chip adapted for use with the techniques described herein for the measurement of biomarker levels in a sample.

In some embodiments, a kit can include a control and/or a reference (e.g., a reference antibody and/or a reference sample from a healthy subject).

In certain embodiments, kits disclosed herein can comprise solid supports and/or compositions for use in methods of determining the concentration of one or more biomarkers in a biological sample (e.g., serum), comprising one or more antigen-binding molecules (e.g., an antibody or antigen-binding fragment thereof) that bind specifically to the one or more biomarkers described above and herein. In certain embodiments, the solid supports and/or compositions comprise an antigen-binding molecule specific for OLR1, ox-LDL, 4-HNE, CCL2 MDA and/or IL18. The solid supports and compositions can optionally also comprise an antigen binding-molecule specific for at least one other biomarker, such as Nf-L, pNf-H, sCD14, LBP, CRP, IL-6, IL-17F, IL-17C, or any combination thereof. Examples of solid supports for use in the kits disclosed herein include, but are not limited to, multiwell plates, slides, beads, and chips. As described above, these solid supports can be used in multiplex immunoassays (e.g., ELISA) to determine the level of one or more biomarkers in a biological sample (e.g., serum).

### 6.3 ALS Therapies

For the methods provided herein such as, for instance, methods of selecting an ALS patient for an ALS therapy, methods for predicting an ALS patient's likelihood of responding to an ALS therapy, methods for monitoring efficacy of an ALS therapy, and the like, the ALS therapy can be any therapy that is administered to an ALS patient to treat ALS. In certain embodiments, provided herein are methods of administering one or more agents that treats or prevents ALS (e.g., based on the agent's product label or known to one skilled in the art e.g., Mead R J et al., Nat Rev Drug Discov. 2022 Dec. 21:1-28). In certain embodiments, an agent that treats and/or prevents ALS can be an immunotherapy, a cell-based therapy, or a combination thereof. In some embodiments, the ALS therapy is a Treg therapy (see Section 6.3.1).

In certain embodiments, methods provided herein can comprise administering the ALS therapy to the ALS patient to treat ALS, e.g., to mitigate a symptom of ALS. In certain embodiments, methods provided herein can comprise administering the ALS therapy to the ALS patient in a clinical trial to test the ALS therapy.

In certain embodiments, an agent to be used according to the methods disclosed herein can be an FDA approved drug indicated for the treatment of ALS. Non-limiting examples of such an agent can include: Relyvrio™ (AMX 0035; a 3:1 combination of sodium phenylbutyrate (4-phenyl butyric acid sodium salt) and taurursodiol (2-[(3α, 7β-dihydroxy-24-oxo-5β-cholan-24-yl) amino]ethane sulfonic acid, dihydrate) also known as tauroursodeoxycholic acid or TUDCA); edaravone (3-methyl-1-phenyl-2-pyrazolin-5-one]; and/or riluzole (2-amino-6-(trifluoromethoxy)benzothiazole).

In certain embodiments, non-limiting examples of an agent that can be used according to the methods disclosed herein for the treatment of ALS can include Tocilizumab, ACTH/Acthar gel, IL-2, Ibudilast, Pentoxifylline, Masitinib, NP001, Thalidomide, Glatiramer Acetate, CC100, Fingolimod, Minocycline, Pioglitazone, DNL747, Fasudil, Ono-2506, Anakinra, RNS60, Retigabine, Memantine, Talampanel, Perampanel, Mexiletine, Ceftriaxone, Celecoxib, Lamotrigine, Nimodipine, Gabapentin, Epi-589, N-Acetylcysteine, Vitamine E, Coenzyme Q10, Selegiline, Rasagiline, Cu(II)-ATSM, BIIB067, Pyrimethamine, Levosimendan, Tirasemtiv, CK-2127107IGF-1, Xaliproden, EPO, BDNF, GM604, CNTF, SB509, VM202, sNN0029, KP-100, Dexpramipexole, Olexosime, Creatine, Acetyl-L-carnitine, Lithium, Rapamycin, Tamoxifen, Ozanezumab, GDC-0134, YAM80, Methylcobalamin, Pimozide, Omigapil/TCH346, Deferiprone, Arimoclomol, EH301, BIIB078, and/or MD1003 (see e.g., Wobst H J et al., Med Res Rev. 2020 July; 40(4): 1352-1384).

In certain embodiments, an agent to be used according to the methods disclosed herein can be a drug for controlling one or more symptoms of ALS. Non-limiting examples of such agents can include topiramate (2,3:4,5-Bis-O-(1-methylethylidene)-beta-D-fructopyranose sulfamate); gabapentin (2-[1-(aminomethyl)cyclohexyl]acetic acid); lamotrigine (6-(2,3-dichlorophenyl)-1,2,4-triazine-3,5-diamine); talampanel ((8R)-7-Acetyl-5-(4-aminophenyl)-8,9-dihydro-8-methyl-7H-1,3-dioxolo[4,5-h] [2,3]benzodiazepine); ceftriaxone ((6R,7R)-7-[[(2Z)-2-(2-amino-1,3-thiazol-4-yl)-2-methoxyiminoacetyl]amino]-3-[(2-methyl-5,6-dioxo-1H-1,2,4-triazin-3-yl)sulfanylmethyl]-8-ox o-5-thia-1-azabicyclo[4.2.0]oct-2-ene-2-carboxylic acid); an inhibitor of glutamate carboxypeptidase II; dextromethorphan/quinidine; TUDCA (tauroursodeoxycholic acid); baclofen (4-amino-3-(4-chlorophenyl)butanoic acid); diazepam (7-chloro-1-methyl-5-phenyl-3H-1,4-benzodiazepin-2-one); trihexyphenidyl (1-cyclohexyl-1-phenyl-3-piperidin-1-ylpropan-1-ol); and/or amitriptyline (N, N-dimethyl-3-(2-tricyclo[9.4.0.03,8]pentadeca-1(15),3,5,7,11,13-hexaenylidene)propan-1-amine).

In certain embodiments, the methods provided herein can comprise administering to the subject a drug that has been repurposed for the treatment of ALS (e.g., edaravone, rasagiline, AMX 0035, masitinib, and/or acrimonclomol). See also Kiernan et al., Nat Rev Neurol. 2021 Feb; 17(2): 104-118.

In certain embodiments, additional non-limiting examples of an agent to be used according to the methods disclosed herein can include abacavir, dolutegravir, lamivudine, curcumin, sodium phenylbutyrate, naltrexone, tauroursodeoxycholic acid (TUDCA), methylcobalamin, dextromethorphan hydrobromide, quinidine sulfate, nudexta, ropinirol, metformin, tamoxifen, trimetazidine, masitinib, CuATSM (diacetylbis(N(4)-methylthiosemicarbazonato)copper(II)), and/or reldesemtiv. See also Pridmore W, (2021). RRNMF Neuromuscular Journal, 2(1), 36-40.

In certain embodiments, an ALS therapy can, for example, comprise TIGLUTIK (thickened riluzole), EXSERVAN™ (riluzole oral film), BHV-0223 (sublingual riluzole), NUEDEXTA® (dextromethorphan HBr and quinidine sulfate), ravulizumab-cwvz (ULTOMIRIS®), mesenchymal stem cell (MSC)-neurotrophic factor (NTF) cells (e.g., NUROWN®), MASITINIB (an oral tyrosine kinase inhibitor), TOFERSEN (BIIB067, IONIS-SOD1Rx, Ionis Pharmaceuticals and Biogen), APB-102 (Apic Bio), H.P. ACTHAR GEL (Mallinckrodt Pharmaceuticals), MN-166 (MediciNova), GM-6 (Genervon), GILENYA (fingolimod, ALS TDI), ARIMOCLOMOL (Orph-001,Orphazyme), NP001 (Neuraltus), VM202 (VM Biopharma), RELDESEMTIV (Cytokinetics), NUROWN (BrainStorm Cell Therapeutics), NSI-566 (Neuralstem), MEXILETINE, acamprosate, baclofen, cinacalcet, sulfisoxazole, torasemide, or any combination thereof.

In certain embodiments of the methods provided herein, the ALS therapy comprises interleukin-2 (“IL-2”) which can be administered alone or in combination with a second agent. In certain embodiments, the IL-2 is aldesleukin. In some embodiments, the ALS therapy is IL-2 and CTLA-4 fusion protein, e.g., abatacept. Exemplary methods of administering IL-2 and abatacept combination therapy are described, for example, in International Application No. PCT/US2022/019748, which is incorporated herein by reference for its teaching of such methods.

### 6.3.1 Treg Therapy

In certain embodiments of the methods provided herein, the ALS therapy is a T regulatory cell (“Treg”) therapy. In certain embodiments, the ALS therapy comprises a Treg infusion. In certain embodiments, the ALS therapy comprises a plurality of Treg infusions.

Administering Treg therapy to ALS patients has been shown to slow progression rates of the disease, and Treg suppressive function in some ALS patients correlates with the slowing of disease progression (Thonhoff, J.R. et al., Neurology-Neuroimmunology Neuroinflammation 5(4): e465 (2018)). However, as demonstrated in the Examples below, not all ALS patients are responsive to Treg therapy. In some embodiments, methods are provided herein to stratify patients that will likely be, or are, responsive to Treg therapy (“responders”), and those that will likely not be, or are not, responsive to Treg therapy (“non-responders”).

Certain methods provided herein may be performed with any steps of isolating, expanding, and administering Treg therapy to subjects (e.g., ALS patients) that are known in the art or disclosed herein. Exemplary methods of producing obtaining, enriching for and ex-vivo expanding a population of Tregs are described in International PCT Application No. PCT/US2020/063378, which is incorporated by reference herein in its entirety, in particular for its teaching of such methods.

As a non-limiting example, in some embodiments, the ALS therapy comprises collecting white blood cells from the ALS patient (leukapheresis), isolating and expanding ex vivo Tregs from the collected white blood cells; and administering the expanded Tregs intravenously (infusion) to the ALS patient.

In certain embodiments of the methods provided herein, a single Treg infusion is administered to the ALS patient. In some embodiments of the methods provided herein, a plurality of Treg infusions are administered to the ALS patient.

In particular embodiments, the Tregs may be administered with IL-2. In certain embodiments, IL-2 is administered then followed by Treg administration. In some embodiments, Treg administration (e.g., infusion) can be administered concomitantly with IL-2, e.g., with subcutaneous IL-2 injection(s).

In some embodiments the ALS therapy comprises anti-inflammatory extracellular vesicles (EVs) derived from ex vivo-expanded Tregs. Exemplary methods for preparing Treg EVs, and for administering Treg EV therapy to patients, are described, for example, in International Application No. PCT/US2022/017990, which is incorporated herein in its entirety, in particular for its teaching of such compositions and methods.

### 6.4 Patient Populations

ALS, also known as Lou Gehrig disease, is a fatal disorder characterized by progressive skeletal muscle weakness and wasting or atrophy (i.e., amyotrophy), spasticity, and fasciculations as a result of degeneration of the UMNs and LMNs, culminating in respiratory paralysis. In general, ALS is classified as sporadic, familial, or Western Pacific ALS with or without Parkinsonism-dementia complex (ALS/PDC). Most ALS cases are sporadic, and only 5-10% of cases are considered to be familial. Mutations in the C9orf72 gene are responsible for 30-40% of familial ALS cases in the United States and Europe. Worldwide, approximately 20% of cases of familial ALS are due to a mutation in the Cu/Zn superoxide dismutase-1 gene (SODI).

Described herein are methods for treating or preventing ALS in a subject suspected of having or diagnosed with ALS. The term “subject” and “patient” are used interchangeably throughout the present disclosure. Subjects for whom the methods provided herein can be used can be diagnosed and selected for based on assessing diagnostic criteria associated with ALS according to the methods described in Section 6.6.1. In certain embodiments, subjects for whom the methods provided herein can be used may have a particular stage of ALS at the time of treatment. Methods of determining disease stage are detailed in Section 6.6.2 below.

In certain embodiments, a subject to the methods disclosed herein is a mammal. In some embodiments, the subject is a human subject. In some embodiments, the subject is an adult human subject. In some embodiments, the subject is a female human subject. In some embodiments, the subject is a male human subject. In some embodiments, the human subject is between about 18 to about 90 years of age. In some embodiments, the human subject is over 65, over 70, over 75, over 80, over 85, or over 90 years of age.

In certain embodiments, a subject to the methods disclosed herein has been diagnosed has having ALS according to Gold Coast criteria (see Section 6.6.1(c)).

In certain embodiments, subjects in need of treatment disclosed herein can include subjects diagnosed as having possible, laboratory supported probable, probable, or definite ALS according to revised El Escorial criteria (see Section 6.6.1(a)). In some embodiments, a subject to the methods for treating or preventing ALS disclosed herein can be a subject diagnosed as having possible ALS. In some embodiments, a subject to the methods for treating or preventing ALS disclosed herein can be a subject diagnosed as having laboratory supported probable ALS. In some embodiments, a subject to the methods for treating or preventing ALS disclosed herein can be a subject diagnosed as having probable ALS. In some embodiments, a subject to the methods for treating or preventing ALS disclosed herein can be a subject diagnosed as having definite ALS.

In certain embodiments, subjects in need of treatment disclosed herein can include subjects diagnosed as having clinically definite, clinically probable, or clinically possible ALS according to Awaji shima criteria (See Section 6.6.1(b)) In some embodiments, a subject to the methods for treating or preventing ALS disclosed herein can be a subject diagnosed as having clinically definite ALS. In some embodiments, a subject to the methods for treating or preventing ALS disclosed herein can be a subject diagnosed as having clinically probable ALS. In some embodiments, a subject to the methods for treating or preventing ALS disclosed herein can be a subject diagnosed as having clinically possible ALS.

In some embodiments, the subject is a human subject recently diagnosed with ALS (e.g., diagnosed as having first symptoms within 3 months to 3 years from start of administration of an ALS therapy contemplated in the present disclosure). In some embodiments, the subject is a human subject diagnosed as having first symptoms within 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months from the start of administration of one or more ALS therapies disclosed herein.

In some embodiments, the human subject is a naïve subject (i.e., a subject that has not been treated for ALS). In some embodiments, the human subject has been previously treated with an agent that is used for treating ALS. In some embodiments, the human subject was not responsive to previous ALS treatment(s). In some embodiments, the subject is unresponsive to at least one treatment for ALS. In some embodiments, the subject is unresponsive to several treatments of ALS. In some embodiments, the subject has sporadic ALS. In some embodiments, the subject has familial ALS. In some embodiments, the subject has a genetic mutation associated with ALS (e.g., mutation in C9orf72, SOD1, TARDBP and/or FUS). In some embodiments, the subject does not have another acute or chronic disorder of the immune system (e.g., only ALS). In some embodiments, the subject does not have another immune related disorder (e.g., only ALS). In some embodiments, the subject does not have another chronic or acute inflammatory disorder of the immune system (e.g., only ALS). In some embodiments, the subject does not have another disease or condition besides ALS. In some embodiments, the subject has another acute or chronic disorder of the immune system (e.g., in addition to ALS). In some embodiments, the subject has another immune related disorder (e.g., in addition to ALS).

In some embodiments, the subject has another chronic or acute inflammatory disorder of the immune system (e.g., in addition to ALS). In some embodiments, the subject has another disease or condition in addition to ALS.

In certain embodiments, patients for whom the methods provided herein can be used include ALS patients at various disease stages (see Section 6.6.2). In some embodiments, ALS patients for whom the methods provided herein can be used can be staged according to the King's system of staging. In some embodiments, ALS patients for whom the methods provided herein can be used are stage 1 ALS patients, stage 2 ALS patients, stage 3 ALS patients, and/or stage 4 ALS patients according to the King's system of staging. In some embodiments, an ALS patient for whom the methods provided herein can be used is a stage 1 ALS patient according to the King's system of staging. In some embodiments, an ALS patient for whom the methods provided herein can be used is a stage 2 ALS patient according to the King's system of staging. In some embodiments, an ALS patient for whom the methods provided herein can be used is a stage 3 ALS patient according to the King's system of staging. In some embodiments, an ALS patient for whom the methods provided herein can be used is a stage 4 ALS patient according to the King's system of staging. In some preferred embodiments, an ALS patient for whom the methods provided herein can be used is a stage 2 ALS patient according to the King's system of staging. In some other preferred embodiments, an ALS patient for whom the methods provided herein can be used is a stage 1 ALS patient according to the King's system of staging.

In some embodiments, ALS patients for whom the methods provided herein can be used can be staged according to the MiToS system of staging. In some embodiments, ALS patients for whom the methods provided herein can be used are stage 1 ALS patients, stage 2 ALS patients, stage 3 ALS patients, and/or stage 4 ALS patients according to the MiToS system of staging. In some embodiments, an ALS patient for whom the methods provided herein can be used is a stage 0 ALS patient according to the MiToS system of staging, wherein the patient reports one or more physiological symptoms of ALS. In some embodiments, an ALS patient for whom the methods provided herein can be used is a stage 1 ALS patient according to the MiToS system of staging. In some embodiments, an ALS patient for whom the methods provided herein can be used is a stage 2 ALS patient according to the MiToS system of staging. In some embodiments, an ALS patient for whom the methods provided herein can be used is a stage 3 ALS patient according to the MiToS system of staging. In some embodiments, an ALS patient for whom the methods provided herein can be used is a stage 4 ALS patient according to the MiToS system of staging. In some preferred embodiments, an ALS patient for whom the methods provided herein can be used is a stage 2 ALS patient according to the MiToS system of staging. In some other preferred embodiments, an ALS patient for whom the methods provided herein can be used is a stage 1 ALS patient according to the MiToS system of staging.

### 6.5 Methods of ALS Treatment

Provided herein are methods comprising administering one or more ALS therapies (see Section 6.3) to a selected ALS patient to treat ALS. In certain embodiments, methods disclosed herein can be used for selecting an ALS patient for an ALS therapy. In certain embodiments, methods disclosed herein can be used for predicting an ALS patient's likely responsiveness to an ALS therapy. In certain embodiments, methods disclosed herein can be used for monitoring efficacy of an ALS therapy. In certain embodiments, methods disclosed herein can comprise administering the ALS therapy to the ALS patient in a clinical trial to test the ALS therapy.

In certain embodiments, methods of the present disclosure are directed, in part, toward preventing ALS in a subject suspected of having or diagnosed with ALS. As used herein, the terms “prevention” and “preventing” refer to obtaining beneficial or desired results including, but not limited, to prophylactic benefit. For prophylactic benefit, the ALS therapies (e.g., Treg, Treg/IL-2) disclosed herein can be administered according to the methods of treating as provided herein to a subject at risk of developing ALS (e.g., a subject diagnosed as having possible ALS) or to a subject reporting one or more physiological symptoms of ALS, even though a diagnosis of the ALS may not have been made. In some embodiments, the subject has at least one symptom associated with ALS. Non-limiting examples of symptoms of ALS include dyspnea, orthopnea, muscle weakness, dysphagia, and dysarthria.

In certain embodiments, methods of the present disclosure are directed, in part, toward treating ALS in a subject suspected of having or diagnosed with ALS. As used herein and unless otherwise indicated, the terms “treat,” “treating,” “treatment,” and “ameliorating” are used interchangeably herein, and mean an alleviation, in whole or in part, of a disorder, disease or condition, such as ALS, or one or more of the symptoms associated with a disorder, disease, or condition, such as ALS, or slowing or halting of further progression or worsening of those symptoms, or alleviating or eradicating the cause(s) of the disorder, disease, or condition itself, such as ALS.

In certain embodiments provided herein, the methods of the present disclosure can be used for selecting/characterizing a patient for amyotrophic lateral sclerosis (ALS) therapy. In some embodiments, a method for selecting a patient for ALS therapy comprises determining whether a concentration of at least one biomarker described herein in a biological sample (e.g., serum) collected from a patient diagnosed with or suspected of having ALS is less than, equal to, or greater than, a baseline or a reference concentration. Exemplary methods of determining a biomarker concentration are detailed in Section 6.1 of the present disclosure.

In some embodiments, methods for selecting a patient for ALS therapy can comprise determining whether a concentration of at least one biomarker described herein in a biological sample (e.g., serum) is less than, equal to, or greater than, a baseline concentration. In particular embodiments, the at least one biomarker is OLR1, ox-LDL, sCD14, LBP, CRP, 4-HNE, IL-6, IL-17F, IL-17C, MDA, CCL2, or IL-18; e.g., OLR1, ox-LDL, sCD14, LBP, CRP, 4-HNE, IL-17F, IL-17C, MDA, CCL2, or IL-18; e.g., OLR1, ox-LDL, sCD14, LBP, CRP, 4-HNE, IL-17F, IL-17C, CCL2, or IL-18; e.g., ox-LDL, 4-HNE, MDA, CCL2, or IL-18; e.g., ox-LDL, 4-HNE, CCL2, or IL-18; e.g., 4-HNE, MDA, CCL2, or IL-18; e.g., 4-HNE, CCL2, or IL-18; e.g., OLR1, ox-LDL, 4-HNE, CCL2, or IL-18; e.g., OLR1, ox-LDL, or 4-HNE; e.g., OLR1 or ox-LDL; e.g., CCL2 or IL-18; or, e.g., OLR1, ox-LDL, CCL2 or IL-18.

In some embodiments, methods for selecting a patient for ALS therapy can comprise determining whether a concentration of at least one biomarker described herein in a biological sample (e.g., serum) is less than, equal to, or greater than, a reference concentration. In particular embodiments, the at least one biomarker is OLR1, ox-LDL, sCD14, LBP, CRP, 4-HNE, IL-6, IL-17F, IL-17C, MDA, CCL2, or IL-18; e.g., OLR1, ox-LDL, sCD14, LBP, CRP, 4-HNE, IL-17F, IL-17C, MDA, CCL2, or IL-18; e.g., OLR1, ox-LDL, sCD14, LBP, CRP, 4-HNE, IL-17F, IL-17C, CCL2, or IL-18; e.g., ox-LDL, 4-HNE, MDA, CCL2, or IL-18; e.g., ox-LDL, 4-HNE, CCL2, or IL-18; e.g., 4-HNE, MDA, CCL2, or IL-18; e.g., 4-HNE, CCL2, or IL-18; e.g., OLR1, ox-LDL, 4-HNE, CCL2, or IL-18; e.g., OLR1, ox-LDL, or 4-HNE; e.g., OLR1 or ox-LDL; e.g., CCL2 or IL-18; or, e.g., OLR1, ox-LDL, CCL2 or IL-18.

In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18, or, e.g., 4-HNE, CCL2, or IL-18, in a biological sample (e.g., serum) is equal to or less than a reference concentration, the patient is selected for ALS therapy. In some embodiments, when a concentration of at least two biomarkers described herein, for example, OLR1, ox-LDL, 4-HNE, CCL2, MDA or IL-18, e.g., OLR1, ox-LDL, 4-HNE, CCL2, or IL-18, or, e.g., 4-HNE, CCL2, or IL-18, in a biological sample (e.g., serum) is equal to or less than the reference concentration, the patient is selected for ALS therapy. In some embodiments, when a concentration of at least three biomarkers described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is equal to or less than the reference concentration, the patient is selected for ALS therapy. In some embodiments, when a concentration of at least four biomarkers described herein, for example, OLR1, ox-LDL, 4-HNE, CCL2, MDA or IL-18, e.g., OLR1, ox-LDL, 4-HNE, CCL2, or IL-18; e.g., OLR1, 4-HNE, CCL2, or IL-18; or, e.g., ox-LDL, 4-HNE, CCL2 or IL-18, in a biological sample (e.g., serum) is equal to or less than the reference concentration, the patient is selected for ALS therapy. In some embodiments, when the concentration of OLR1, ox-LDL, 4-HNE, CCL2, and IL-18 in a biological sample (e.g., serum) is equal to or less than the reference concentration, the patient is selected for ALS therapy.

In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is up to about 0.5-fold less, up to about 1-fold less, up to about 1.5-fold less, up to about 2-fold less, up to about 2.5-fold less, up to about 3-fold less, up to about 3.5-fold less, up to about 4-fold less, up to about 4.5-fold less, up to about 5- fold less, up to about 6-fold less, up to about 6.5-fold less, up to about 7-fold less, up to about 7.5-fold less, up to about 8-fold less, up to about 8.5-fold less, up to about 9-fold less, up to about 9.5-fold less, up to about 10-fold less, or more than up to about 10-fold less than a reference concentration, the patient is selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is at least about 0.5-fold less, at least about 1-fold less, at least about 1.5-fold less, at least about 2-fold less, at least about 2.5-fold less, at least about 3-fold less, at least about 3.5-fold less, at least about 4-fold less, at least about 4.5-fold less, at least about 5- fold less, at least about 6-fold less, at least about 6.5-fold less, at least about 7-fold less, at least about 7.5-fold less, at least about 8-fold less, at least about 8.5-fold less, at least about 9-fold less, at least about 9.5-fold less, at least about 10-fold less, or more than at least about 10-fold less than the reference concentration, the patient is selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is between about 0.5-fold less to about 10-fold less, between about 0.5-fold less to about 1-fold less, between about 1-fold less to about 1.5-fold less, between about 1.5-fold less to about 2-fold less, between about 2-fold less to about 2.5-fold less, between about 2.5-fold less to about 3-fold less, between about 3-fold less to about 3.5-fold less, between about 3.5-fold less to about 4-fold less, between about 4-fold less to about 4.5-fold less, between about 4.5-fold less to about 5-fold less, between about 5-fold less to about 5.5-fold less, between about 5.5-fold less to about 6-fold less, between about 6-fold less to about 6.5-fold less, between about 6.5-fold less to about 7-fold less, between about 7-fold less to about 7.5-fold less, between about 7.5-fold less to about 8-fold less, between about 8-fold less to about 8.5-fold less, between about 8.5-fold less to about 9-fold less, between about 9-fold less to about 9.5-fold less, or between about 9.5-fold less to about 10-fold less than the reference concentration, the patient is selected for ALS therapy.

In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is up to about 1% less, up to about 2% less, up to about 3% less, up to about 4% less, up to about 5% less, up to about 6% less, up to about 7% less, up to about 8% less, up to about 9% less, up to about 10% less, up to about 12% less, about 14% less, up to about 16% less, up to about 18% less, up to about 20% less, up to about 25% less, up to about 30% less, up to about 35% less, up to about 40% less, up to about 45% less, or up to about 50% less than a reference concentration, the patient is selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is at least about 1% less, at least about 2% less, at least about 3% less, at least about 4% less, at least about 5% less, at least about 6% less, at least about 7% less, at least about 8% less, at least about 9% less, at least about 10% less, at least about 12% less, at least about 14% less, at least about 16% less, at least about 18% less, at least about 20% less, at least about 25% less, at least about 30% less, at least about 35%, less at least about 40% less, at least about 45% less, or at least about 50% less than the reference concentration, the patient is selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is between about 1% less to about 50% less, between about 10% less to about 50% less, between about 10% less to about 40% less, between about 1% less to about 2% less, between about 2% less to about 3% less, between about 3% less to about 4% less, between about 4% less to about 5% less, between about 5% less to about 6% less, between about 6% less to about 7% less, between about 7% less to about 8% less, between about 8% less to about 9% less, between about 9% less to about 10% less, between about 10% less to about 12% less, between about 12% less to about 14% less, between about 14% less to about 16% less, between about 16% less to about 18% less, between about 18% less to about 20% less, between about 20% less to about 25% less, between about 25% less to about 30% less, between about 35% less to about 40% less, between about 40% less to about 45% less, or between about 45% less to about 50% less than the reference concentration, the patient is selected for ALS therapy.

In certain embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is greater than a reference concentration, the patient is not selected for ALS therapy. In some embodiments, when a concentration of at least two biomarkers described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is greater than the reference concentration, the patient is not selected for ALS therapy. In some other embodiments, when a concentration of at least three biomarkers described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is greater than the reference concentration, the patient is not selected for ALS therapy. In some other embodiments, when a concentration of at least four biomarkers described herein, for example, OLR1, ox-LDL, 4-HNE, CCL2, MDA or IL-18, e.g., OLR1, ox-LDL, 4-HNE, CCL2, or IL-18; e.g., OLR1, 4-HNE, CCL2, or IL-18; or, e.g., ox-LDL, 4-HNE, CCL2, or IL-18, in a biological sample (e.g., serum) is greater than the reference concentration, the patient is not selected for ALS therapy. In some other embodiments, when a concentration of OLR1, ox-LDL, 4-HNE, CCL2, and IL-18 in a biological sample (e.g., serum) is greater than their reference concentrations, the patient is not selected for ALS therapy. In some other embodiments, when a concentration of 4-HNE, CCL2, and IL-18 in a biological sample (e.g., serum) is greater than their reference concentrations, the patient is not selected for ALS therapy.

In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is up to about 0.5-fold greater, up to about 1-fold greater, up to about 1.5-fold greater, up to about 2-fold greater, up to about 2.5-fold greater, up to about 3-fold greater, up to about 3.5-fold greater, up to about 4-fold greater, up to about 4.5-fold greater, up to about 5-fold greater, up to about 6-fold greater, up to about 6.5-fold greater, up to about 7-fold greater, up to about 7.5-fold greater, up to about 8-fold greater, up to about 8.5-fold greater, up to about 9-fold greater, up to about 9.5-fold greater, up to about 10-fold greater, or more than up to about 10-fold greater than a reference concentration, the patient is not selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is at least about 0.5-fold greater, at least about 1-fold greater, at least about 1.5-fold greater, at least about 2-fold greater, at least about 2.5-fold greater, at least about 3-fold greater, at least about 3.5-fold greater, at least about 4-fold greater, at least about 4.5-fold greater, at least about 5-fold greater, at least about 6-fold greater, at least about 6.5-fold greater, at least about 7-fold greater, at least about 7.5-fold greater, at least about 8-fold greater, at least about 8.5-fold greater, at least about 9-fold greater, at least about 9.5-fold greater, at least about 10-fold greater, or more than at least about 10-fold greater than the reference concentration, the patient is not selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is between about 0.5-fold greater to about 10-fold greater, between about 0.5-fold greater to about 1-fold greater, between about 1-fold greater to about 1.5-fold greater, between about 1.5-fold greater to about 2-fold greater, between about 2-fold greater to about 2.5-fold greater, between about 2.5-fold greater to about 3-fold greater, between about 3-fold greater to about 3.5-fold greater, between about 3.5-fold greater to about 4-fold greater, between about 4-fold greater to about 4.5-fold greater, between about 4.5-fold greater to about 5-fold greater, between about 5-fold greater to about 5.5-fold greater, between about 5.5-fold greater to about 6-fold greater, between about 6-fold greater to about 6.5-fold greater, between about 6.5-fold greater to about 7-fold greater, between about 7-fold greater to about 7.5-fold greater, between about 7.5-fold greater to about 8-fold greater, between about 8-fold greater to about 8.5-fold greater, between about 8.5-fold greater to about 9-fold greater, between about 9-fold greater to about 9.5-fold greater, or between about 9.5-fold greater to about 10-fold greater than the reference concentration, the patient is not selected for ALS therapy.

In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is up to about 1% greater, up to about 2% greater, up to about 3% greater, up to about 4% greater, up to about 5% greater, up to about 6% greater, up to about 7% greater, up to about 8% greater, up to about 9% greater, up to about 10% greater, up to about 12% greater, about 14% greater, up to about 16% greater, up to about 18% greater, up to about 20% greater, up to about 25% greater, up to about 30% greater, up to about 35% greater, up to about 40% greater, up to about 45% greater, or up to about 50% greater than a reference concentration, the patient is not selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is at least about 1% greater, at least about 2% greater, at least about 3% greater, at least about 4% greater, at least about 5% greater, at least about 6% greater, at least about 7% greater, at least about 8% greater, at least about 9% greater, at least about 10% greater, at least about 12% greater, at least about 14% greater, at least about 16% greater, at least about 18% greater, at least about 20% greater, at least about 25% greater, at least about 30% greater, at least about 35%, greater at least about 40% greater, at least about 45% greater, or at least about 50% greater than the reference concentration, the patient is not selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18, in a biological sample (e.g., serum) is between about 1% greater to about 50% greater, between about 10% greater to about 50% greater, between about 10% greater to about 40% greater, between about 1% greater to about 2% greater, between about 2% greater to about 3% greater, between about 3% greater to about 4% greater, between about 4% greater to about 5% greater, between about 5% greater to about 6% greater, between about 6% greater to about 7% greater, between about 7% greater to about 8% greater, between about 8% greater to about 9% greater, between about 9% greater to about 10% greater, between about 10% greater to about 12% greater, between about 12% greater to about 14% greater, between about 14% greater to about 16% greater, between about 16% greater to about 18% greater, between about 18% greater to about 20% greater, between about 20% greater to about 25% greater, between about 25% greater to about 30% greater, between about 35% greater to about 40% greater, between about 40% greater to about 45% greater, or between about 45% greater to about 50% greater than the reference concentration, the patient is not selected for ALS therapy.

In some embodiments, a method for selecting a patient for ALS therapy comprises determining whether a concentration of at least one biomarker in a biological sample (e.g., serum) collected from a patient diagnosed with or suspected of having ALS is different than baseline. Methods of determining baseline are detailed in Section 6.1 of the present disclosure. In some embodiments, methods for selecting a patient for ALS therapy can comprise determining whether a concentration of at least one biomarker in a biological sample (e.g., serum) is different than baseline wherein the at least one biomarker is described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, a method for selecting a patient who has been administered at least one for ALS therapy for at least one additional ALS therapy comprises determining whether a concentration of at least one biomarker in a biological sample (e.g., serum) collected from a patient diagnosed with or suspected of having ALS is different than baseline. In some embodiments, a method for selecting a patient who has been administered at least one ALS therapy for a different ALS therapy comprises determining whether a concentration of at least one biomarker in a biological sample (e.g., serum) collected from a patient diagnosed with or suspected of having ALS is different than baseline. In some embodiments, a method for selecting a patient who has been administered at least one ALS therapy for another dosage of that ALS therapy comprises determining whether a concentration of at least one biomarker in a biological sample (e.g., serum) collected from a patient diagnosed with or suspected of having ALS is different than baseline. In some embodiments, a method for selecting a patient who has been administered at least one ALS therapy for another dosage of that same ALS therapy in addition to an additional ALS therapy comprises determining whether a concentration of at least one biomarker in a biological sample (e.g., serum) collected from a patient diagnosed with or suspected of having ALS is different than baseline. In some embodiments, a method for selecting a patient who has been administered at least one ALS therapy for another administration of that same ALS therapy at a different dosage (e.g., a higher dosage, a lower dosage) comprises determining whether a concentration of at least one biomarker in a biological sample (e.g., serum) collected from a patient diagnosed with or suspected of having ALS is different than baseline.

In some embodiments, methods for selecting a patient for ALS therapy can comprise determining whether a concentration of at least two biomarkers in a biological sample (e.g., serum) is different than baseline wherein the at least two biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least three biomarkers in a biological sample (e.g., serum) is different than baseline wherein the at least three biomarkers are described herein, for example, OLR1, ox-LDL, 4-HNE, CCL2, MDA or IL-18, e.g., OLR1, ox-LDL, 4-HNE, CCL2 or IL-18; or, e.g., 4-HNE, CCL2, or IL18. In some embodiments, methods for selecting a patient for ALS therapy can comprise determining whether a concentration of at least four biomarkers in a biological sample (e.g., serum) is different than baseline wherein the at least four biomarkers are described herein, for example, OLR1, ox-LDL, 4-HNE, CCL2, MDA or IL-18, e.g., OLR1, ox-LDL, 4-HNE, CCL2, or IL-18; or, e.g., OLR1, 4-HNE, CCL2, or IL-18; or, e.g., ox-LDL, 4-HNE, CCL2, or IL18. In some embodiments, methods for selecting a patient for ALS therapy can comprise determining whether a concentration of biomarkers in a biological sample (e.g., serum) is different than baseline wherein the biomarkers are OLR1, ox-LDL, 4-HNE, CCL2, and IL-18. In some embodiments, methods for selecting a patient for ALS therapy can comprise determining whether a concentration of biomarkers in a biological sample (e.g., serum) is different than baseline wherein the biomarkers are 4-HNE, CCL2, and IL-18.

In some embodiments, methods for selecting a patient for ALS therapy can comprise determining whether a concentration of at least one biomarker in a biological sample (e.g., serum) is higher than baseline wherein the at least one biomarker is described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least two biomarkers in a biological sample (e.g., serum) is higher than baseline wherein the at least two biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least three biomarkers in a biological sample (e.g., serum) is higher than baseline wherein the at least three biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least four biomarkers in a biological sample (e.g., serum) is higher than baseline wherein the at least four biomarkers are described herein, for example, OLR1, ox-LDL, 4-HNE, CCL2, MDA or IL-18, e.g., OLR1, ox-LDL, 4-HNE, CCL2, or IL-18; e.g., OLR1, 4-HNE, CCL2 or IL-18; or, e.g., ox-LDL, 4-HNE, CCL2, or IL-18. In some embodiments, methods for selecting a patient for ALS therapy can comprise determining whether a concentration of biomarkers in a biological sample (e.g., serum) is higher than baseline wherein the biomarkers are OLR1, ox-LDL, 4-HNE, CCL2, and IL-18. In some embodiments, methods for selecting a patient for ALS therapy can comprise determining whether a concentration of biomarkers in a biological sample (e.g., serum) is higher than baseline wherein the biomarkers are 4-HNE, CCL2, and IL-18.

In some embodiments, methods for selecting a patient for ALS therapy can comprise determining whether a concentration of at least one biomarker in a biological sample (e.g., serum) is lower than baseline wherein the at least one biomarker is described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least two biomarkers in a biological sample (e.g., serum) is lower than baseline wherein the at least two biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least three biomarkers in a biological sample (e.g., serum) is lower than baseline wherein the at least three biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least four biomarkers in a biological sample (e.g., serum) is lower than baseline wherein the at least four biomarkers are described herein, for example, OLR1, ox-LDL, 4-HNE, CCL2, MDA or IL-18, e.g., OLR1, ox-LDL, 4-HNE, CCL2, or IL-18; e.g., OLR1, 4-HNE, CCL2 or IL-18; or, e.g., ox-LDL, 4-HNE, CCL2, or IL-18. In some embodiments, methods for selecting a patient for ALS therapy can comprise determining whether a concentration of biomarkers in a biological sample (e.g., serum) is lower than baseline wherein the biomarkers are OLR1, ox-LDL, 4-HNE, CCL2, and IL-18. In some embodiments, methods for selecting a patient for ALS therapy can comprise determining whether a concentration of biomarkers in a biological sample (e.g., serum) is lower than baseline wherein the biomarkers are 4-HNE, CCL2, and IL-18.

In some embodiments, methods for selecting a patient for ALS therapy can comprise determining whether a concentration of at least one biomarker in a biological sample (e.g., serum) is lower than baseline and whether a concentration of at least one other biomarker is higher than baseline wherein the biomarker is described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least one biomarker in a biological sample (e.g., serum) is higher than baseline and whether a concentration of at least one other biomarker is lower than baseline wherein the biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18.

In some embodiments, methods for selecting a patient for ALS therapy can comprise determining whether a concentration of at least two biomarkers in a biological sample (e.g., serum) are lower than baseline and whether a concentration of at least one biomarker other than those two is higher than baseline wherein the biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least two biomarkers in a biological sample (e.g., serum) are higher than baseline and whether a concentration of at least one biomarker other than those two is lower than baseline wherein the biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least two biomarkers in a biological sample (e.g., serum) are higher than baseline and whether a concentration of at least two other biomarkers are lower than baseline wherein the biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18.

In some embodiments, methods for selecting a patient for ALS therapy can comprise determining whether a concentration of at least three biomarkers in a biological sample (e.g., serum) are lower than baseline and whether a concentration of at least one biomarker other than those three is higher than baseline wherein the biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least three biomarkers in a biological sample (e.g., serum) are higher than baseline and whether a concentration of at least one biomarker other than those three is lower than baseline wherein the biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18.

In some embodiments, methods for selecting a patient for ALS therapy can comprise determining whether a concentration of at least four biomarkers in a biological sample (e.g., serum) are lower than baseline and whether a concentration of at least one biomarker other than those four is higher than baseline wherein the biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18. In some embodiments, methods for selecting a patient for ALS therapy can comprise determining whether a concentration of at least four biomarkers in a biological sample (e.g., serum) are higher than baseline and whether a concentration of at least one biomarker other than those four is lower than baseline wherein the biomarkers are described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL18.

In certain embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is lower than baseline, the patient is selected for ALS therapy. In some other embodiments, when a concentration of at least two biomarkers described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is lower than baseline, the patient is selected for ALS therapy. In some other embodiments, when a concentration of at least three biomarkers described herein, for example, OLR1, ox-LDL, 4-HNE, CCL2, MDA or IL-18, e.g., OLR1, Ox-LDL, 4-HNE, CCL2, or IL-18, or, e.g., 4-HNE, CCL2, or IL-18 in a biological sample (e.g., serum) is lower than baseline, the patient is selected for ALS therapy. In some other embodiments, when a concentration of at least four biomarkers described herein, for example, OLR1, ox-LDL, 4-HNE, CCL2, MDA or IL-18, e.g., OLR1, ox-LDL, 4-HNE, CCL2, or IL-18; e.g., ORL1, 4-HNE, CCL2, or IL-18; or, e.g., ox-LDL, 4-HNE, CCL2, or IL-18 in a biological sample (e.g., serum) is lower than baseline, the patient is selected for ALS therapy. In some other embodiments, when a concentration of OLR1, ox-LDL, 4-HNE, CCL2, and IL-18 in a biological sample (e.g., serum) is lower than baseline, the patient is selected for ALS therapy. In some other embodiments, when a concentration of 4-HNE, CCL2, and IL- 18 in a biological sample (e.g., serum) is lower than baseline, the patient is selected for ALS therapy.

In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is up to about 0.5-fold lower, up to about 1-fold lower, up to about 1.5-fold lower, up to about 2-fold lower, up to about 2.5-fold lower, up to about 3-fold lower, up to about 3.5-fold lower, up to about 4-fold lower, up to about 4.5-fold lower, up to about 5-fold lower, up to about 6-fold lower, up to about 6.5-fold lower, up to about 7-fold lower, up to about 7.5-fold lower, up to about 8-fold lower, up to about 8.5-fold lower, up to about 9-fold lower, up to about 9.5-fold lower, up to about 10-fold lower, or more than up to about 10-fold lower than baseline, the patient is selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is at least about 0.5-fold lower, at least about 1-fold lower, at least about 1.5-fold lower, at least about 2-fold lower, at least about 2.5-fold lower, at least about 3-fold lower, at least about 3.5-fold lower, at least about 4-fold lower, at least about 4.5-fold lower, at least about 5-fold lower, at least about 6-fold lower, at least about 6.5-fold lower, at least about 7-fold lower, at least about 7.5-fold lower, at least about 8-fold lower, at least about 8.5-fold lower, at least about 9-fold lower, at least about 9.5-fold lower, at least about 10-fold lower, or more than at least about 10-fold lower than baseline, the patient is selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is between about 0.5-fold lower to about 10-fold lower, between about 0.5-fold lower to about 1-fold lower, between about 1-fold lower to about 1.5-fold lower, between about 1.5-fold lower to about 2-fold lower, between about 2-fold lower to about 2.5-fold lower, between about 2.5-fold lower to about 3-fold lower, between about 3-fold lower to about 3.5-fold lower, between about 3.5-fold lower to about 4-fold lower, between about 4-fold lower to about 4.5-fold lower, between about 4.5-fold lower to about 5-fold lower, between about 5-fold lower to about 5.5-fold lower, between about 5.5-fold lower to about 6-fold lower, between about 6-fold lower to about 6.5-fold lower, between about 6.5-fold lower to about 7-fold lower, between about 7-fold lower to about 7.5-fold lower, between about 7.5-fold lower to about 8-fold lower, between about 8-fold lower to about 8.5-fold lower, between about 8.5-fold lower to about 9-fold lower, between about 9-fold lower to about 9.5-fold lower, or between about 9.5-fold lower to about 10-fold lower than baseline, the patient is selected for ALS therapy.

In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is up to about 1% lower, up to about 2% lower, up to about 3% lower, up to about 4% lower, up to about 5% lower, up to about 6% lower, up to about 7% lower, up to about 8% lower, up to about 9% lower, up to about 10% lower, up to about 12% lower, about 14% lower, up to about 16% lower, up to about 18% lower, up to about 20% lower, up to about 25% lower, up to about 30% lower, up to about 35% lower, up to about 40% lower, up to about 45% lower, or up to about 50% lower than baseline, the patient is selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is at least about 1% lower, at least about 2% lower, at least about 3% lower, at least about 4% lower, at least about 5% lower, at least about 6% lower, at least about 7% lower, at least about 8% lower, at least about 9% lower, at least about 10% lower, at least about 12% lower, at least about 14% lower, at least about 16% lower, at least about 18% lower, at least about 20% lower, at least about 25% lower, at least about 30% lower, at least about 35%, lower at least about 40% lower, at least about 45% lower, or at least about 50% lower than baseline, the patient is selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is between about 1% lower to about 50% lower, between about 10% lower to about 50% lower, between about 10% lower to about 40% lower, between about 1% lower to about 2% lower, between about 2% lower to about 3% lower, between about 3% lower to about 4% lower, between about 4% lower to about 5% lower, between about 5% lower to about 6% lower, between about 6% lower to about 7% lower, between about 7% lower to about 8% lower, between about 8% lower to about 9% lower, between about 9% lower to about 10% lower, between about 10% lower to about 12% lower, between about 12% lower to about 14% lower, between about 14% lower to about 16% lower, between about 16% lower to about 18% lower, between about 18% lower to about 20% lower, between about 20% lower to about 25% lower, between about 25% lower to about 30% lower, between about 35% lower to about 40% lower, between about 40% lower to about 45% lower, or between about 45% lower to about 50% lower than baseline, the patient is selected for ALS therapy.

In certain embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is higher than baseline, the patient is not selected for ALS therapy. In some other embodiments, when a concentration of at least two biomarkers described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is higher than baseline, the patient is not selected for ALS therapy. In some other embodiments, when a concentration of at least three biomarkers described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is higher than baseline, the patient is not selected for ALS therapy. In some other embodiments, when a concentration of at least four biomarkers described herein, for example, OLR1, ox-LDL, 4-HNE, CCL2, MDA or IL-18, e.g., OLR1, ox-LDL, 4-HNE, CCL2, or IL-18; e.g., ORL1, 4-HNE, CCL2, or IL-18; or, e.g., 4-HNE, CCL2, or IL-18 in a biological sample (e.g., serum) is higher than baseline, the patient is not selected for ALS therapy. In some other embodiments, when a concentration of OLR1, ox-LDL, 4-HNE, CCL2, and IL-18 in a biological sample (e.g., serum) is higher than baseline, the patient is not selected for ALS therapy. In some other embodiments, when a concentration of 4-HNE, CCL2, and IL-18 in a biological sample (e.g., serum) is higher than baseline, the patient is not selected for ALS therapy.

In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is up to about 0.5-fold higher, up to about 1-fold higher, up to about 1.5-fold higher, up to about 2-fold higher, up to about 2.5-fold higher, up to about 3-fold higher, up to about 3.5-fold higher, up to about 4-fold higher, up to about 4.5-fold higher, up to about 5-fold higher, up to about 6-fold higher, up to about 6.5-fold higher, up to about 7-fold higher, up to about 7.5-fold higher, up to about 8-fold higher, up to about 8.5-fold higher, up to about 9-fold higher, up to about 9.5-fold higher, up to about 10-fold higher, or more than up to about 10-fold higher than baseline, the patient is not selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is at least about 0.5-fold higher, at least about 1-fold higher, at least about 1.5-fold higher, at least about 2-fold higher, at least about 2.5-fold higher, at least about 3-fold higher, at least about 3.5-fold higher, at least about 4-fold higher, at least about 4.5-fold higher, at least about 5-fold higher, at least about 6-fold higher, at least about 6.5-fold higher, at least about 7-fold higher, at least about 7.5-fold higher, at least about 8-fold higher, at least about 8.5-fold higher, at least about 9-fold higher, at least about 9.5-fold higher, at least about 10-fold higher, or more than at least about 10-fold higher than baseline, the patient is not selected for ALS therapy. In some embodiments, when a concentration of at least one biomarker described herein, for example, OLR1, ox-LDL, 4-HNE, CCL2, MIDA or IL-18, e.g., OLR1, ox-LDL, 4-HNE, CCL2, or IL-18, or, e.g., 4-HNE, CCL2, or IL-18 in a biological sample (e.g., serum) is between about 0.5-fold higher to about 10-fold higher, between about 0.5-fold higher to about 1-fold higher, between about 1-fold higher to about 1.5-fold higher, between about 1.5-fold higher to about 2-fold higher, between about 2-fold higher to about 2.5-fold higher, between about 2.5-fold higher to about 3-fold higher, between about 3-higher to about 3.5-fold higher, between about 3.5-fold higher to about 4-fold higher, between about 4-fold higher to about 4.5-fold higher, between about 4.5-fold higher to about 5-fold higher, between about 5-fold higher to about 5.5-fold higher, between about 5.5-fold higher to about 6-fold higher, between about 6-fold higher to about 6.5-fold higher, between about 6.5-fold higher to about 7-fold higher, between about 7-fold higher to about 7.5-fold higher, between about 7.5-fold higher to about 8-fold higher, between about 8-fold higher to about 8.5-fold higher, between about 8.5-fold higher to about 9-fold higher, between about 9-fold higher to about 9.5-fold higher, or between about 9.5-fold higher to about 10-fold higher than baseline, the patient is not selected for ALS therapy.

In some embodiments, when a concentration of at least one biomarker described herein, for example, OLR1, ox-LDL, 4-HNE, CCL2, MDA or IL-18 e.g., OLR1, ox-LDL, 4-HNE, CCL2, or IL-18; or, e.g., 4-HNE, CCL2, or IL-18 in a biological sample (e.g., serum) is up to about 1% higher, up to about 2% higher, up to about 3% higher, up to about 4% higher, up to about 5% higher, up to about 6% higher, up to about 7% higher, up to about 8% higher, up to about 9% higher, up to about 10% higher, up to about 12% higher, about 14% higher, up to about 16% higher, up to about 18% higher, up to about 20% higher, up to about 25% higher, up to about 30% higher, up to about 35% higher, up to about 40% higher, up to about 45% higher, or up to about 50% higher than baseline, the patient is not selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is at least about 1% higher, at least about 2% higher, at least about 3% higher, at least about 4% higher, at least about 5% higher, at least about 6% higher, at least about 7% higher, at least about 8% higher, at least about 9% higher, at least about 10% higher, at least about 12% higher, at least about 14% higher, at least about 16% higher, at least about 18% higher, at least about 20% higher, at least about 25% higher, at least about 30% higher, at least about 35%, higher at least about 40% higher, at least about 45% higher, or at least about 50% higher than baseline, the patient is not selected for ALS therapy. In some embodiments, when a concentration of at least one biomarker described herein, for example, OLR1, ox-LDL, 4-HNE, CCL2, MDA or IL-18, e.g., OLR1, ox-LDL, 4-HNE, CCL2, or IL-18; or, e.g., 4-HNE, CCL2, or IL-18 in a biological sample (e.g., serum) is between about 1% higher to about 50% higher, between about 10% higher to about 50% higher, between about 10% higher to about 40% higher, between about 1% higher to about 2% higher, between about 2% higher to about 3% higher, between about 3% higher to about 4% higher, between about 4% higher to about 5% higher, between about 5% higher to about 6% higher, between about 6% higher to about 7% higher, between about 7% higher to about 8% higher, between about 8% higher to about 9% higher, between about 9% higher to about 10% higher, between about 10% higher to about 12% higher, between about 12% higher to about 14% higher, between about 14% higher to about 16% higher, between about 16% higher to about 18% higher, between about 18% higher to about 20% higher, between about 20% higher to about 25% higher, between about 25% higher to about 30% higher, between about 35% higher to about 40% higher, between about 40% higher to about 45% higher, or between about 45% higher to about 50% higher than baseline, the patient is not selected for ALS therapy.

Also provided herein are methods of monitoring efficacy of an ALS therapy (e.g., a Treg therapy) to treat ALS. In certain embodiments, a method of monitoring efficacy of an ALS therapy comprises: (a) administering an ALS therapy to a patient diagnosed with ALS; (b) determining whether a concentration of at least one biomarker in a serum sample collected from the patient after administration of the ALS therapy is less than, equal to, or greater than, a reference concentration, wherein the at least one biomarker comprises: OLR1, ox-LDL, 4-HNE, CCL2, and IL-18; (c) wherein the ALS therapy has poor efficacy where the concentration of the at least one biomarker is greater than its reference concentration. In certain embodiments, a method of monitoring efficacy of an ALS therapy comprises: (a) administering an ALS therapy to a patient diagnosed with ALS; (b) determining whether a concentration of at least one biomarker in a serum sample collected from the patient after administration of the ALS therapy is less than, equal to, or greater than, a reference concentration, wherein the at least one biomarker comprises: 4-HNE, CCL2, and IL-18; (c) wherein the ALS therapy has poor efficacy where the concentration of the at least one biomarker is greater than its reference concentration.

In some embodiments, the ALS therapy has poor efficacy where the concentration of the at least one biomarker is more than about 0.5-fold greater, more than about 1-fold greater, more than about 2-fold greater, more than about 3-fold greater, more than about 4-fold greater, more than about 5-fold greater, more than about 6-fold greater, more than about 7-fold greater, more than about 8-fold greater, more than about 9-fold greater, more than about 10-fold greater, or more than about 20-fold greater than its reference concentration. In some embodiments, the ALS therapy has poor efficacy where the concentration of the at least one biomarker is more than between about 1-fold and about 20-fold greater than its reference concentration, more than between about 1-fold and about 10-fold greater than its reference concentration, more than between about 1-fold and about 5-fold greater than its reference concentration, more than between about 5-fold and about 10-fold greater than its reference concentration, more than between about 5-fold and about 20-fold greater than its reference concentration, or more than between about 10-fold and about 20-fold greater than its reference concentration.

In some embodiments, the ALS therapy has poor efficacy where the concentration of the at least one biomarker is more than about 1% greater, more than about 5% greater, more than about 10% greater, more than about 15% greater, more than about 20% greater, more than about 25% greater, more than about 30% greater, more than about 35% greater, more than about 40% greater, or more than about 50% greater than its reference concentration. In some embodiments, the ALS therapy has poor efficacy where the concentration of the at least one biomarker is more than between about 1% and about 50% greater than its reference concentration, more than between about 1% and about 40% greater than its reference concentration, more than between about 1% and about 30% greater than its reference concentration, more than between about 1% and about 20% greater than its reference concentration, more than between about 1% and about 10% greater than its reference concentration, more than between about 10% and about 50% greater than its reference concentration, more than between about 20% and about 50% greater than its reference concentration, more than between about 30% and about 50% greater than its reference concentration, or more than between about 10% and about 10% greater than its reference concentration.

In certain embodiments, a method of monitoring efficacy of an ALS therapy comprises: (a) administering an ALS therapy to a patient diagnosed with ALS; (b) determining whether a concentration of at least one biomarker in a serum sample collected from the patient after administration of the ALS therapy is less than, equal to, or greater than baseline wherein the at least one biomarker comprises: OLR1, ox-LDL, 4-HNE, CCL2, and IL-18; (c) wherein the ALS therapy has poor efficacy where the concentration of the at least one biomarker is greater than baseline.

In certain embodiments, a method of monitoring efficacy of an ALS therapy comprises: (a) administering an ALS therapy to a patient diagnosed with ALS; (b) determining whether a concentration of at least one biomarker in a serum sample collected from the patient after administration of the ALS therapy is less than, equal to, or greater than baseline wherein the at least one biomarker comprises: 4-HNE, CCL2, and IL-18; (c) wherein the ALS therapy has poor efficacy where the concentration of the at least one biomarker is greater than baseline.

In some embodiments, the ALS therapy has poor efficacy where the concentration of the at least one biomarker is more than about 0.5-fold greater, more than about 1-fold greater, more than about 2-fold greater, more than about 3-fold greater, more than about 4-fold greater, more than about 5-fold greater, more than about 6-fold greater, more than about 7-fold greater, more than about 8-fold greater, more than about 9-fold greater, more than about 10-fold greater, or more than about 20-fold greater than baseline. In some embodiments, the ALS therapy has poor efficacy where the concentration of the at least one biomarker is more than between about 1-fold and about 20-fold greater than baseline, more than between about 1-fold and about 10-fold greater than baseline, more than between about 1-fold and about 5-fold greater than baseline, more than between about 5-fold and about 10-fold greater than baseline, more than between about 5-fold and about 20-fold greater than baseline, or more than between about 10-fold and about 20-fold greater than baseline.

In some embodiments, the ALS therapy has poor efficacy where the concentration of the at least one biomarker is more than about 1% greater, more than about 5% greater, more than about 10% greater, more than about 15% greater, more than about 20% greater, more than about 25% greater, more than about 30% greater, more than about 35% greater, more than about 40% greater, or more than about 50% greater than baseline. In some embodiments, the ALS therapy has poor efficacy where the concentration of the at least one biomarker is more than between about 1% and about 50% greater than baseline, more than between about 1% and about 40% greater than baseline, more than between about 1% and about 30% greater than baseline, more than between about 1% and about 20% greater than baseline, more than between about 1% and about 10% greater than baseline, more than between about 10% and about 50% greater than baseline, more than between about 20% and about 50% greater than baseline, more than between about 30% and about 50% greater than baseline, or more than between about 10% and about 10% greater than baseline.

In certain embodiments, a method of treating ALS comprises: (a) administering an ALS therapy (e.g., a Treg infusion) to a patient diagnosed with ALS; (b) comparing a concentration of at least one biomarker in a serum sample obtained from the patient after the administration to a reference concentration/baseline, wherein the at least one immune-based biomarker comprises OLR1, ox-LDL, 4-HNE, CCL2, and IL-18; and (c) administering an ALS therapy comprising a plurality of doses to the patient if the concentration of the at least one biomarker is equal to or below the reference concentration/baseline. In certain embodiments, a method of treating ALS comprises: (a) administering an ALS therapy (e.g., a Treg infusion) to a patient diagnosed with ALS; (b) comparing a concentration of at least one biomarker in a serum sample obtained from the patient after the administration to a reference concentration/baseline, wherein the at least one immune-based biomarker comprises 4-HNE, CCL2, and IL-18; and (c) administering an ALS therapy comprising a plurality of doses to the patient if the concentration of the at least one biomarker is equal to or below the reference concentration/baseline. For the present disclosure, the term “reference concentration/baseline” refers to standards for methods that can be performed by either assessing the concentration of at least one biomarker in a serum sample as compared to its reference concentration as detailed above or by assessing the concentration of at least one biomarker in a serum sample as compared to baseline as detailed above.

In certain embodiments, the method comprises: (a) administering to the patient an ALS therapy (e.g., a Treg infusion) comprising doses being administered to the patient on different days; (b) comparing concentrations of at least one biomarker in serum samples to a reference concentration/baseline, wherein each of the serum sample is obtained from the patient after a dose of the ALS therapy, wherein the at least one biomarker comprises OLR1, ox-LDL, 4-HNE, CCL2, and IL-18; and (c) maintaining the patient on the ALS therapy comprising administering doses following step (b), if the concentrations of the at least biomarker is at, or less than the reference concentration/baseline in all or at least about 50% (e.g., about 1%, 5%, 10%, 15%, 20%, 25%, 30%, 35%, 40%, 45%, 50%) of the serum samples.

In certain embodiments, the method comprises: (a) administering to the patient an ALS therapy (e.g., a Treg infusion) comprising doses being administered to the patient on different days; (b) comparing concentrations of at least one biomarker in serum samples to a reference concentration/baseline, wherein each of the serum sample is obtained from the patient after a dose of the ALS therapy, wherein the at least one biomarker comprises 4-HNE, CCL2, and IL-18; and (c) maintaining the patient on the ALS therapy comprising administering doses following step (b), if the concentrations of the at least biomarker is at, or less than the reference concentration/baseline in all or at least about 50% (e.g., about 1%, 5%, 10%, 15%, 20%, 25%, 30%, 35%, 40%, 45%, 50%) of the serum samples.

In certain embodiments, provided herein is a method for treating ALS in a patient diagnosed therewith, the method comprising: (a) determining whether the concentration of at least one biomarker in a serum sample obtained from a patient diagnosed with ALS is at or below a reference concentration/baseline of the at least one biomarker, wherein the serum sample is obtained from the patient after being administered with an ALS therapy (e.g., a Treg infusion), wherein the at least one biomarker comprises OLR1, ox-LDL, 4-HNE, CCL2, and IL-18 and (b) administering to the patient an ALS therapy comprising a plurality of doses if the concentration of the at least one biomarker is determined to be at or below its reference concentration/baseline.

In certain embodiments, provided herein is a method for treating ALS in a patient diagnosed therewith, the method comprising: (a) determining whether the concentration of at least one biomarker in a serum sample obtained from a patient diagnosed with ALS is at or below a reference concentration/baseline of the at least one biomarker, wherein the serum sample is obtained from the patient after being administered with an ALS therapy (e.g., a Treg infusion), wherein the at least one biomarker comprises 4-HNE, CCL2, and IL-18 and (b) administering to the patient an ALS therapy comprising a plurality of doses if the concentration of the at least one biomarker is determined to be at or below its reference concentration/baseline.

In certain embodiments, provided herein are methods for predicting a patient's likely responsiveness to an ALS therapy. In some embodiments, the method comprises (a) collecting a serum sample from a patient diagnosed with ALS; and (b) comparing a concentration of at least one biomarker to a reference concentration/baseline; wherein the patient is predicted as likely to be non-responsive to the ALS therapy where the concentration of the at least one biomarker is determined to be greater than its reference concentration/baseline.

In certain embodiments, the method further comprises enrolling the patient in a clinical trial to test an ALS therapy if the concentration of the at least one biomarker is the same or less than its reference concentration/baseline or is in or below its reference concentration range/baseline range.

In certain embodiments, the method further comprises administering an ALS therapy to the patient if the concentration of the at least one serum immune-based biomarker is the same or less than its reference concentration/baseline or is in or below its reference concentration range/baseline range.

In certain embodiments, if the concentration of the at least one biomarker is greater than its reference concentration/baseline, the patient is predicted to be non-responsive to ALS therapy. In some embodiments, if the concentration of the at least one biomarker is more than about 0.5-fold greater, more than about 1-fold greater, more than about 2-fold greater, more than about 3-fold greater, more than about 4-fold greater, more than about 5-fold greater, more than about 6-fold greater, more than about 7-fold greater, more than about 8-fold greater, more than about 9-fold greater, more than about 10-fold greater, or more than about 20-fold greater than its reference concentration/baseline, the patient is predicted as likely to be non-responsive to ALS therapy.

In certain embodiments, provided herein is a method of assessing the likely progression of ALS in a patient diagnosed with ALS. In some embodiments, the method comprises comparing a concentration of at least one biomarker in a serum sample obtained from the patient diagnosed with ALS to a reference concentration and assessing the likely progression of ALS to be fast if the concentration of the at least one serum immune-based biomarker is elevated relative to a reference concentration, and assessing the likely progression of ALS to be slow if the concentration of the at least biomarker is at or below its referenced concentration. In certain embodiments, one or more of the biomarkers disclosed herein can indicates a particular disease state (e.g., slowly progressing ALS, rapidly progressing ALS).

In certain embodiments, methods disclosed herein can determine if a subject diagnosed with ALS that is assessed to have rapid progression is treatment responsive. In some embodiments, an ALS patient who has rapidly progressing ALS is not treatment responsive if one or more biomarkers does not change after administration of a first ALS treatment. In some embodiments, an ALS patient who has rapidly progressing ALS is not treatment responsive if the concentration of CCL-2 levels does not change after administration of a first ALS treatment. In some embodiments, an ALS patient who has rapidly progressing ALS is not treatment responsive if the concentration of IL-18 levels does not change after administration of a first ALS treatment. In some embodiments, an ALS patient who has rapidly progressing ALS is not treatment responsive if the concentration of CCL-2 and IL-18 levels does not change after administration of a first ALS treatment.

In certain embodiments, the method further comprises administering an ALS therapy to the patient if the ALS is assessed to have fast progression. In certain embodiments provided herein are methods for treating an ALS patient comprising administering an ALS therapy to the ALS patient; assessing the responsiveness of the ALS patient to the ALS therapy; and (a) continue administering the ALS therapy to the ALS patient if the ALS patient is assessed to be responsive to the ALS therapy, or (b) discontinue administering the ALS therapy to the ALS patient if the ALS patient is assessed to be non-responsive to the ALS therapy; wherein the responsiveness of the ALS patient to the ALS therapy comprises comparing the serum concentration of an biomarker to a reference concentration/baseline and the ALS patient is assessed to be responsive to the ALS therapy if the serum concentration of the biomarker decreases or remains within the reference concentration/baseline in successive serum samples taken from the ALS patient, otherwise the ALS patient is assessed to be non-responsive to the ALS therapy.

In certain embodiments, the responsiveness of the ALS patient to the ALS therapy comprises comparing the serum concentration of an biomarker to a reference concentration/baseline for each biomarker in a set of two or more biomarkers, and the ALS patient is assessed to be responsive to the ALS therapy if the serum concentration of each biomarker in the set of two or more biomarkers decreases or remains within the reference concentration/baseline in successive serum samples taken from the ALS patient, otherwise the ALS patient is assessed to be non-responsive to the ALS therapy. In some embodiments, the set of two or more biomarkers comprises 2, 3, 4, 5, 6, 7, 8, 9, or 10 biomarkers. In some embodiments, the set of two or more biomarkers comprises OLR1, ox-LDL, 4-HNE, CCL2, and/or IL-18. In some embodiments, the set of two or more biomarkers comprises 4-HNE, CCL2, and/or IL-18.

In certain embodiments, the responsiveness of the ALS patient to the ALS therapy comprises comparing the serum concentration of a biomarker to a reference concentration/baseline for each biomarker in a set of biomarkers, and the ALS patient is assessed to be responsive to the ALS therapy if the serum concentration of at least about 50% (e.g., about 1%, 5%, 10%, 15%, 20%, 25%, 30%, 35%, 40%, 45%, 50%) of the biomarkers in the set of biomarkers decreases or remains within the reference concentration/baseline in successive serum samples taken from the ALS patient, otherwise the ALS patient is assessed to be non-responsive to the ALS therapy. In some embodiments, the set of biomarkers comprises 1, 2, 3, 4, 5, 6, 7, 8, 9, or 10 biomarkers. In some embodiments, the set of biomarkers comprises OLR1, ox-LDL, 4-HNE, CCL2, and/or IL-18. In some embodiments, the set of biomarkers comprises 4-HNE, CCL2, and/or IL-18.

It will be understood that “successive serum samples” means serum samples taken from the ALS patient on different days, e.g., on different visits to a hospital, clinician's office or wherever blood samples are taken from the ALS patient. For example, “at least one successive serum sample” refers to a serum sample taken on a day following the day on which in initial or first serum sample was taken from the ALS patient. In certain embodiments, successive serum samples comprise at least two serum samples. In certain embodiments, successive serum samples comprise at least three serum samples. In certain embodiments, successive serum samples comprise at least four serum samples. In certain embodiments, successive serum samples comprise at least five serum samples. In certain embodiments, successive serum samples comprise at least six serum samples. In certain embodiments, successive serum samples comprise at least seven serum samples. The successive serum samples can, for example, be consecutive. In some embodiments, the successive serum samples are non-consecutive.

In certain embodiments, the ALS patient is assessed to be responsive to the ALS therapy if the serum concentration of the biomarker decreases or remains within the reference concentration/baseline in a majority of 3 or more successive serum samples taken from the ALS patient. It will be understood that, in certain embodiments, when assessing responsiveness of the ALS patient to an ALS therapy by comparing serum concentration of each of one or more biomarkers to a reference concentration/baseline for each of the one or more biomarkers in successive serum samples, the ALS patient can be assessed to be responsive to the ALS therapy if a majority of the successive serum samples show decrease in concentration relative to a concentration in an initial serum sample, or if a majority of successive serum samples remain with a reference concentration/baseline.

In certain embodiments, a serum sample is taken the same day on which an ALS therapy is administered to the ALS patient. In certain embodiments, a serum sample is taken 1, 2, 3, 4, 5, 6 or 7 days after the day on which an ALS therapy is administered to the ALS patient. In certain embodiments, the initial serum sample taken after an ALS therapy is administered is taken the same day on which an ALS therapy is administered to the ALS patient. In certain embodiments, the initial serum sample is a serum sample taken after an ALS therapy is administered is taken 1, 2, 3, 4, 5, 6 or 7 days after the day on which an ALS therapy is administered to the ALS patient. In certain embodiments, one or more successive serum samples are taken 1, 2, 3, 4, 5, 6 or 7 days, one week, two weeks, three weeks, or four weeks apart.

In certain embodiments provided herein is a method for treating an ALS patient comprising administering a first ALS therapy to the ALS patient; assessing the responsiveness of the ALS patient to the first ALS therapy; and (a) continue administering the first ALS therapy to the ALS patient if the ALS patient is assessed to be responsive to the first ALS therapy, or (b) discontinue administering the first ALS therapy to the ALS patient if the ALS patient is assessed to be non-responsive to the first ALS therapy and administer a second ALS therapy to the patient, wherein the first and second ALS therapies are different ALS therapies; wherein the responsiveness of the ALS patient to the first ALS therapy comprises comparing the serum concentration of a biomarker to a reference concentration/baseline and the ALS patient is assessed to be responsive to the first ALS therapy if the serum concentration of the biomarker decreases or remains within the reference concentration/baseline in successive serum samples taken from the ALS patient, otherwise the ALS patient is assessed to be non-responsive to the first ALS therapy.

In certain embodiments, the first and/or second ALS therapy comprises administering Relyvrio™ (AMX0035), edaravone, riluzole, or any combination thereof to the patient. In certain embodiments, the first and/or second ALS therapy comprises administering one or more Treg infusions to the patient. In certain embodiments, the first and/or second ALS therapy comprises administering one or more Treg extracellular vesicle (EV), e.g., exosome, infusions to the patient. In certain embodiments, the first and/or second ALS therapy comprises administering a CTLA-4 fusion protein and IL-2 to the patient. In some embodiments, the CTLA-4 fusion protein is abatacept. In certain embodiments, the IL-2 is aldesleukin.

In certain embodiments, the first and/or second ALS therapy comprises administering a gene therapy to the patient. In some embodiments, a gene therapy for treating ALS according to the methods disclosed herein can be one that targets one or more gene mutations associated with ALS. In some embodiments, a gene therapy for treating ALS according to the methods disclosed herein can be one that targets one or more mutations in the genes SOD1, C9orf72, and/or FUS.

In certain embodiments, the first and/or second ALS therapy comprises administering an anti-inflammatory agent. Non-limiting examples of anti-inflammatory agents include aspirin, non-aspirin nonsteroidal anti-inflammatory drugs (NSAIDs), acetaminophen, and the like. In certain embodiments, the first and/or second ALS therapy comprises administering an antioxidant agent. Non-limiting examples of anti-antioxidant agents include riluzole, edaravone, masitinib, (R)-troloxamide quinone, phytochemicals, and the like.

In certain embodiments, provided herein is a method for treating ALS in a patient diagnosed therewith, the method comprising: administering a first ALS therapy to the ALS patient; assessing the responsiveness of the ALS patient to the first ALS therapy; and continuing to administer the first ALS therapy to the ALS patient if the ALS patient is assessed to be responsive to the first ALS therapy, wherein the responsiveness of the ALS patient to the ALS therapy comprises comparing the serum concentration of at least one biomarker to a reference concentration/baseline of the of least one biomarker, wherein the ALS patient is assessed to be responsive to the first ALS therapy if the serum concentration of the biomarker decreases or remains within the reference concentration/baseline in at least one successive serum sample taken from the ALS patient.

In certain embodiments of the above method for treating ALS, in instances where a patient is found to not be responsive to the first ALS therapy, the method further comprises administering to the patient a second ALS therapy. In some embodiments, the method may further comprise, following the administering the second ALS therapy to the ALS patient, assessing the responsiveness of the ALS patient to the second ALS therapy, and continuing to administer the second ALS therapy to the ALS patient if the ALS patient is assessed to be responsive to the second ALS therapy, wherein the responsiveness of the ALS patient to the ALS therapy comprises comparing the serum concentration of at least one biomarker to a reference concentration/baseline of the of least one biomarker, wherein the ALS patient is assessed to be responsive to the second ALS therapy if the serum concentration of the biomarker decreases or remains within the reference concentration/baseline in at least one successive serum sample taken from the ALS patient.

In certain embodiments provided herein are methods for treating an ALS patient comprising administering a combination of ALS therapies to the ALS patient; assessing the responsiveness of the ALS patient to the combination of ALS therapies administered; and continue administering the combination of ALS therapies to the ALS patient if the ALS patient is assessed to be responsive to the combination of ALS therapies; wherein the responsiveness of the ALS patient to the combination of ALS therapies comprises comparing the serum concentration of one or more biomarkers to a reference concentration/baseline and the ALS patient is assessed to be responsive to the ALS therapy if the serum concentration of the one or more biomarker decreases or remains within its respective reference concentration/baseline in successive serum samples taken from the ALS patient.

In certain embodiments of the methods provided herein, the responsiveness of the ALS patient to an ALS therapy, or to a combination of ALS therapies, comprises comparing the serum concentration of a plurality of immune-based biomarkers to their reference concentrations/baseline, and the ALS patient is assessed to be response to the ALS therapy, or combination of ALS therapies, if the serum concentration of the plurality of biomarkers decrease or remains within their respective reference concentration/baseline in successive serum samples. In some embodiments, the plurality of biomarkers comprises 2, 3, 4, 5, 6, 7, 8, 9 or 10 biomarkers. In some embodiments, the plurality of biomarkers comprises OLR1, ox-LDL, 4-HNE, CCL2, and/or IL-18. In some embodiments, the plurality of biomarkers comprises 4-HNE, CCL2, and/or IL-18.

In yet other embodiments of a method provided herein for treating an ALS patient with an ALS therapy (e.g., Treg therapy), the method further comprises performing an additional therapeutic intervention. An additional therapeutic intervention can, for example, be ventilator use, wheelchair use, breathing care, physical therapy (e.g., to address pain, walking, mobility, low-impact exercises), occupational therapy, speech therapy, percutaneous endoscopic gastrostomy (PEG), and palliative care (e.g., medical management for muscle spasms, hypersalivation, pseudobulbar affect, cognitive impairment, and depression).

In some embodiments, provided herein are methods of treating an ALS patient comprising administering an ALS therapy to the patient, wherein the ALS therapy comprises administering IL-2, e.g., aldesleukin, and CTLA-4 fusion protein, e.g., abatacept, as a combination therapy to the patient, assessing responsiveness of the ALS patient to the ALS therapy, wherein (a) when the concentration of a based biomarker decreases over a period of time of 2, 3, 4, 5, 6, 7, 8, 9, 10, 11, 12, or more weeks during which the ALS therapy was administered to the ALS patient, or when the concentration of the biomarker remains elevated within a reference concentration range/baseline over a period of time of 2, 3, 4, 5, 6, 7, 8, 9, 10, 11, 12, or more weeks during which the ALS therapy was administered to the ALS patient, the patient is responsive to the ALS therapy; and (b) when the concentration of the biomarker increases over a period of time of 2, 3, 4, 5, 6, 7, 8, 9, 10, 11, 12, or more weeks during which the ALS therapy was administered to the ALS patient, or when the concentration of the serum-immune based biomarker remains elevated over a reference concentration/baseline range over a period of time of 2, 3, 4, 5, 6, 7, 8, 9, 10, 11, 12, or more weeks during which the ALS therapy was administered to the ALS patient, the patient is non-responsive to the ALS therapy; and, optionally, further administering the ALS therapy to the ALS patient where the ALS patient is assessed to be responsive to the ALS therapy. The biomarker can, for example, be any biomarker described herein. In some embodiments, the biomarker is OLR1. In some other embodiments, the biomarker is ox-LDL. In still some other embodiments, the biomarker is 4-HNE. In some embodiments, the biomarker is CCL2. In some other embodiments, the biomarker is IL-18.

In some embodiments, provided herein are methods of treating an ALS patient comprising administering an ALS therapy (e.g., Treg EV) to the patient, assessing responsiveness of the ALS patient to the ALS therapy, wherein (a) when the concentration of a biomarker decreases over a period of time of 2, 3, 4, 5, 6, 7, 8, 9, 10, 11, 12, or more weeks during which the ALS therapy was administered to the ALS patient, or when the concentration of the biomarker remains elevated within a reference concentration range/baseline over a period of time of 2, 3, 4, 5, 6, 7, 8, 9, 10, 11, 12, or more weeks during which the ALS therapy was administered to the ALS patient, the patient is responsive to the ALS therapy; and (b) when the concentration of the biomarker increases over a period of time of 2, 3, 4, 5, 6, 7, 8, 9, 10, 11, 12, or more weeks during which the ALS therapy was administered to the ALS patient, or when the concentration of the biomarker remains elevated over a reference concentration range/baseline over a period of time of 2, 3, 4, 5, 6, 7, 8, 9, 10, 11, 12, or more weeks during which the ALS therapy was administered to the ALS patient, the patient is non-responsive to the ALS therapy; and, optionally, further administering the ALS therapy to the ALS patient where the ALS patient is assessed to be responsive to the ALS therapy. The biomarker can, for example, be any biomarker described herein. In some embodiments, the biomarker is OLR1. In some other embodiments, the biomarker is ox-LDL. In still some other embodiments, the biomarker is 4-HNE. In some embodiments, the biomarker is CCL2. In some other embodiments, the biomarker is IL-18.

### 6.6 Diagnostic Criteria and Disease Staging

Provided herein are various methods for selecting patients for treatment with the methods disclosed herein. Also provided herein are methods of selecting patients with various stages of ALS to be treated with the methods disclosed herein. Methods of diagnosing subjects to be treated with a method disclosed herein can include assessing diagnostic criteria (Section 6.6.1) in the subject. Methods of determining disease stage are detailed in Section 6.6.2 below.

### 6.6.1 Diagnostic Criteria

Various diagnostic criteria are available for diagnosing and/or predicting the onset of ALS. Exemplary diagnostic criteria for ALS include El Escorial criteria, Awaji Shima criteria, and Gold Coast criteria.

### (a) El Escorial Criteria

An ALS diagnosis may be carried out according to revised El Escorial criteria as described in Ludolph A et al., Amyotroph Lateral Scler Frontotemporal Degener. 2015; 16(5-6): 291-2. According to such analysis, a subject's upper motor neuron (UMN) and lower motor neuron (LMN) impairment is assessed in bulbar, cervical, thoracic, and lumbosacral regions of the body. UMN assessment includes analysis of spasticity and hyper-reflexivity. LMN assessment includes assessment of weakness, atrophy, and fasciculation. Subjects with signs of UMN and LMN impairment in at least three regions tested are diagnosed as having “definite” ALS. Subjects with signs of UMN and LMN impairment in at least two regions tested are diagnosed as having “probable” ALS. Subjects with UMN and LMN signs in one region with evidence by electromyography (EMG) of LMN involvement in another region are characterized as having “laboratory supported probable” ALS. Subjects with signs of UMN and LMN impairment in one region tested or have UMN impairment alone in two or more regions are characterized as having “possible” ALS. Subjects with signs of LMN impairment alone in two or more regions tested are characterized as having “suspected” ALS.

### (b) Awaji Shima criteria

ALS may be diagnosed according to Awaji shima criteria as described in deCarvalho M et al., Clin Neurophysiol. 2008; 119(3): 497-503. Awaji shima criteria integrate electrophysiological criteria with clinical examination findings where the electrophysiological evidence is considered of equal weight to the clinical exam findings of lower motor neuron abnormality. In some examples, Awaji shima criteria and El Escorial criteria can be used in combination to diagnose a subject as having ALS. Electrophysiological criteria are assessed by EMG, a technique known in the art that detects electrical activity in muscles. EMG is useful in detecting the findings of acute denervation (fibrillation and positive sharp waves), chronic denervation (long-duration, complex motor unit action potentials [MUAP]), and chronic reinnervation (large amplitude MUAP).

To be indicative of ALS according to Awaji shima criteria, an EMG generally shows signs of acute or chronic denervation in at least three spinal levels (bulbar, cervical, thoracic, and/or lumbosacral). If three spinal levels are not abnormal, acute or chronic denervation needs to be evident in three extremities with the involvement of at least two muscles supplied by two different roots and two different nerves in each extremity. According to Awaji shima criteria, “clinically definite ALS” is defined by clinical or electrophysiological evidence by the presence of LMN as well as UMN signs in the bulbar region and at least 2 spinal regions or the presence of LMN and UMN signs in 3 spinal regions. “Clinically probable ALS” is defined by clinical or electrophysiological evidence of LMN and UMN signs in at least 2 regions with some UMN signs necessarily rostral to (above) the LMN signs. “Clinically possible ALS” is defined when clinical or electrophysiological signs of UMN and LMN dysfunction are found in only 1 region or UMN signs are found alone in 2 regions or LMN signs are found rostral to UMN signs.

### (c) Gold Coast Criteria

ALS may be diagnosed in a subject according to Gold Coast criteria as described in Shefner J M et al., Clin Neurophysiol 2020 August 131(8): 1975-1978. According to Gold Coast criteria, a subject may be diagnosed as having ALS if: 1) progressive motor impairment was documented by history or repeated clinical assessment, preceded by normal motor function; 2) upper and lower motor neuron dysfunction is present in at least one body region with either (i) upper and lower motor neuron dysfunction noted in the same body region if only one region is involved or (ii) lower motor neuron dysfunction in at least two body regions; and 3) other disease processes are excluded. Upper motor neuron dysfunction implies the presence of at least one of the following: increased deep tendon reflexes, including the presence of a reflex in a clinically weak and wasted muscle, or spread to adjacent muscles; or the presence of pathological reflexes, including Hoffman sign, Babinski sign, crossed adductor reflex, or snout reflex. Lower motor neuron dysfunction in a given muscle requires either: (1) clinical examination evidence of muscle weakness and muscle wasting; or (2) EMG abnormalities that generally include both (i) evidence of chronic neurogenic change, defined by large motor unit potentials of increased duration and/or increased amplitude (with polyphasia) and motor unit instability regarded as supportive but not obligatory evidence, and (ii) evidence of ongoing denervation, including fibrillation potentials or positive sharp waves, or fasciculation potentials.

### 6.6.2 Disease Staging

Various staging systems are available for measuring the severity and/or rate of progression of ALS. Exemplary staging systems include the King's Clinical Staging system and the Milano-Torino Staging (MiToS) system. The MiToS system generally has a higher resolution in late disease, when functional involvement has developed, and the King's system generally has a higher resolution in early disease to mid-disease, when clinical or disease burden is increasing.

The King's system uses five stages, from 1 to 5 and is based on disease burden as measured by clinical involvement and significant feeding or respiratory failure, with stage 1 being symptom onset and stage 5 being death. ALS progression is defined by loss of independence in four key domains on the ALS Functional Rating Scale (ALSFRS): swallowing, walking/self-care, communicating and breathing. The King's system defines the stages as follows: stage 0, functional involvement but no loss of independence on any domain; stages 1-4, number of domains in which independence was lost; and stage 5, death.

The MiToS system involves six stages (0-5) based on functional ability assessed with the ALSFRS-R; stage 0 is defined as normal function and stage 5 is death. The MiTOS stage at diagnosis correlates with survival at 18 months. Stage 1: symptom onset (involvement of first region); Stage 2A: diagnosis; Stage 2B: involvement of second region; Stage 3: involvement of third region; Stage 4A: need for gastrostomy; and Stage 4B: need for non-invasive ventilation.

### 7. EXAMPLES

The following is a description of various methods and materials used in the studies. They are put forth so as to provide those of ordinary skill in the art with a complete disclosure and description of how to make and use the present invention and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### 7.1 Example 1: ALS Therapy: T-Regulatory Cells (Tregs)

### 7.1.1 Patient Selection

Patients with ALS were diagnosed according to the revised El Escorial criteria and the Appel ALS (AALS) score (range 30-164) by an experienced ALS neurologist according to the methods described in Ludolph A et al., Amyotroph Lateral Scler Frontotemporal Degener. 2015; 16(5-6): 291-2 and Haverkamp L J et al., Brain 1995; 118, 707-719, respectively. Rapidly progressing ALS patients were defined as those progressing at a rate of greater than or equal to 1.5 AALS points/month whereas slowly progressing patients progressed at less than 1.5 AALS points/month (Henkel J S et al., EMBO Mol. Med. 2013; 5(1): 64-79). Healthy control (HC) patients were typically spouses and friends of patients, and exclusion criteria included any other neurologic conditions, or autoimmune or infectious diseases. The demographics for the subjects with ALS were (n=30) (mean [SD]) 58.8[1.57] years; 63.3% were men and 36.7% were women; 86.7% were white, 6.7% were Hispanic, 3.3% were black, and 3.3% were Asian. HC individuals (n=10) were similar in age (57.6[2.15 ] years) with 60% men and 40% were women; 90.0% were white, none were Hispanic, none were black, and 10% were Asian.

For the phase I Treg/IL-2 trial, three subjects with ALS were selected based on their differing sites of disease onset and progression rates. Specifically, three patients with arm, bulbar, and leg-onset ALS, respectively, were enrolled in the trial

For the phase IIA Treg/IL-2 trial, eight patients with ALS were enrolled who met the El Escorial criteria for possible, probable, laboratory-supported probable, or definite ALS.

Additional eligibility criteria included a decline in the ALS Functional Rating Scale-Revised (ALSFRS-R) total score of at least 2 points in the 90 days before screening or at least 4 points over the 180 days before screening, a forced vital capacity (FVC)≥65% of predicted capacity at screening, and either no use of riluzole and willingness to refrain from initiating the agent for the duration of the trial or on a stable regimen of riluzole for at least 30 days at the time of screening. Participants who were on edaravone at the time of screening, refrained from receiving edaravone on the same day as the infusion of the study drug. Participants not on edaravone at the time of screening, refrained from initiating the drug for the duration of the trial.

### 7.1.2 Treg Therapy Administration and Patient Monitoring

Subjects with ALS underwent Treg therapy. In brief, Treg therapy included taking T-regulatory cells (Tregs) from the patient (leukapheresis), increasing the cell number in a lab, and returning the Tregs in monthly intravenous (IV) infusions back to the same patient (dose of 1×10⁶ cells/kg), plus three times per week subcutaneous interleukin-2 injections (2×10⁵ IU/m²/injection).

For the phase I Treg/IL-2 trial, patients underwent a total of 8 infusions of expanded autologous Tregs with concomitant subcutaneous IL-2 injections. Four Treg infusions were administered every 2 weeks at an early stage of the disease, followed by 4 Treg infusions administered every 4 weeks at a later stage. IL-2 was administered subcutaneously 3 times weekly beginning the day after the first Treg infusion and continued throughout the study period.

The phase IIA Treg/IL-2 trial was comprised of a 24-week randomized, double-blind, placebo-controlled trial (RCT) followed by a 24-week ascending dose open-label extension (OLE). Tregs and IL-2 were administered during the RCT with Treg/placebo infusions every 4 weeks and the IL-2 subcutaneous injections 3 d/wk. During the OLE, the Treg dose was (1×10⁶ cells/kg/infusion) was administered every 4 weeks for 2 infusions, then double and triple the starting dose for 2 infusions each.

For both trials, patients were closely monitored for adverse effects and changes in disease progression rates. To monitor the responsiveness of the ALS patients to treatment, ALSFRS-R and/or AALS scoring and pulmonary function tests (e.g., FVC) were performed immediately before each Treg infusion, every 2 weeks during each round of infusions, and monthly after each round.

For the eight patients in the phase IIA Treg/IL-2 trial, ALSFRS scores were determined for each patient (Subject Nos. 103, 114, 115, 201, 202, 203, 205, and 206) prior to, and after onset of Treg/IL-2 therapy. Of the eight patients, six (Subject Nos. 114, 115, 202, 203, 205, and 206) were identified as being responsive (“responders”) to Treg infusions by having ALSFRS scores well above the ALSFRS scores of patients administered ceftriaxone (failed in phase 3 clinical trail when administered to ALS patients, see, e.g., Cudkowicz et al., Lancet Neurol. (2014) 13(11): 1083-1091) or to ALSFRS scores reported in PRO-ACT. The two remaining patients (Subject Nos. 103 and 201) were identified as being non-responsive (“non-responders”) to Treg infusions. Subject Nos. 103, 201, and 202 were also rapidly progressing patients according to the criteria described above in Section 7.1.1. See Thonhoff J R et al., Neurol Neuroimmunol Neuroinflamm Nov 2022, 9 (6) e200019, which is incorporated herein by reference for its disclosure of phase IIA Treg/IL-2 trial patient ALSFRS scores.

### 7.2 Example 2: Assessment of Serum Immune-Based Biomarker Concentrations

Serum was obtained from groups of people including those diagnosed with amyotrophic lateral sclerosis (ALS) and healthy volunteers. Serum was collected from blood drawn from a patient according to standard procedures in the art. The resulting serum was subjected to an enzyme-linked immunosorbent assay (ELISA) to assess biomarker (e.g., analyte) concentration in serum of the patient. ELISA was performed according to manufacturer's instructions.

Specifically, serum from patients and/or healthy volunteers are subjected to an ELISA to assess the concentration of one or more of the following analytes: oxidized low-density lipoprotein receptor 1 (OLR1), oxidized low-density lipoprotein (ox-LDL), 4-hydroxynonenal (4-HNE), soluble CD14 (sCD14), lipopolysaccharide binding protein (LBP), C-reactive protein (CRP), interleukin 17F (IL-17F), monocyte chemoattractant protein-1 (CCL2), interleukin-18 (IL-18), 4-malondialdehyde (MDA), neurofilament light chain (Nf-L), and phosphorylated neurofilament heavy chain (pNf-H).

### 7.3 Example 3: Monitoring ALS Disease Progression and Response to ALS Therapy with Biomarkers

Immune-based biomarker concentrations were assessed in serum samples collected from ALS patients and healthy control (HC) subjects according to the methods described in Section 7.2. Concentrations of the biomarkers were determined before and throughout the duration of ALS therapy which was administered according to the methods described in Section 7.1. Specifically, the concentration of inflammatory analytes (CCL2, IL- 18), oxidative stress (OS) analytes (4-HNE and MDA), and analytes associated with neuronal structural integrity (Nf-L and pNf-H) were assessed in serum collected from the patients and HC subjects. As understood herein, an analyte is a substance or chemical constituent that is determined in an analytical procedure. Analytes detected by ELISA, as used in the exemplary methods herein, are typically proteins, for example, cytokines, chemokines, immunoglobulins, hormones, and the like. To analyze the detection and quantification of the target analytes of the present example, comparisons were performed using ANOVA for more than 2 groups or Student's t-test for two groups. ANOVA was presented with the degrees of freedom, F value, and p value and Student's t-test was presented with a p value. Correlation was done using Spearman Rank Order in SigmaStat software and presented with a rho and p values.

### 7.3.1 Inflammatory Analytes

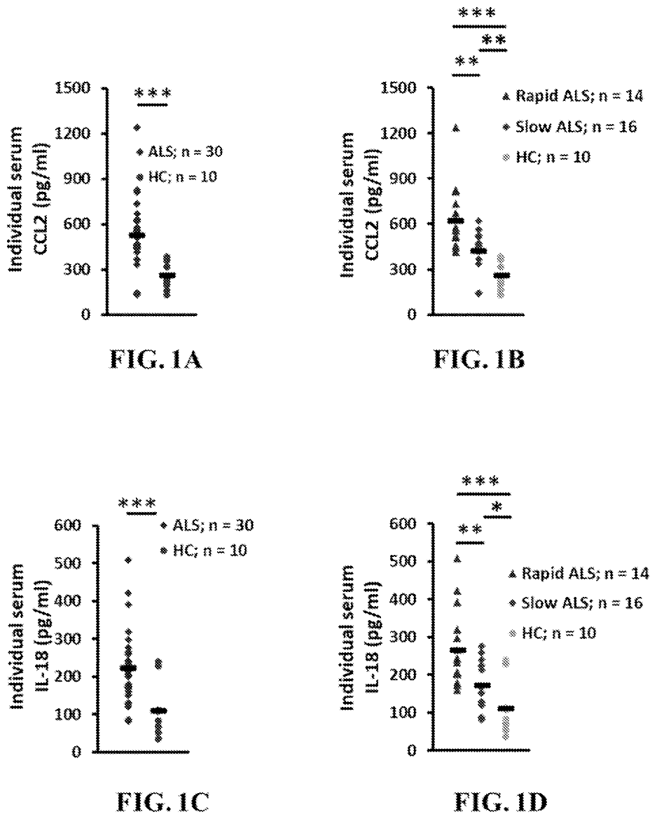

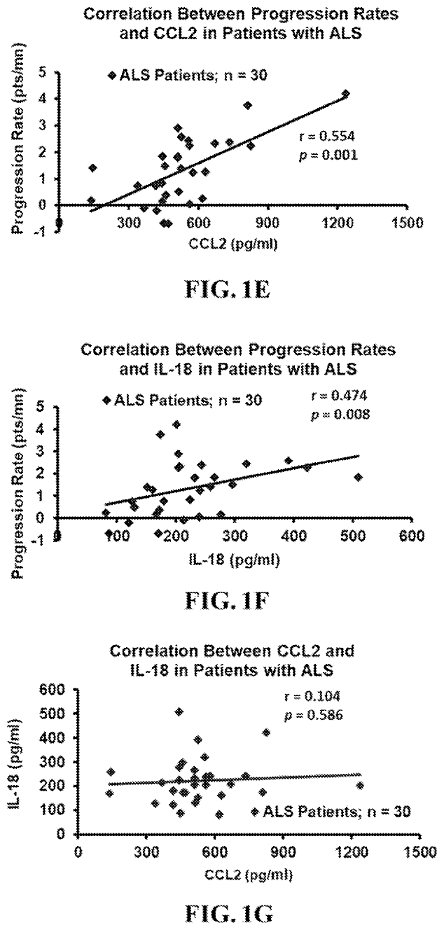

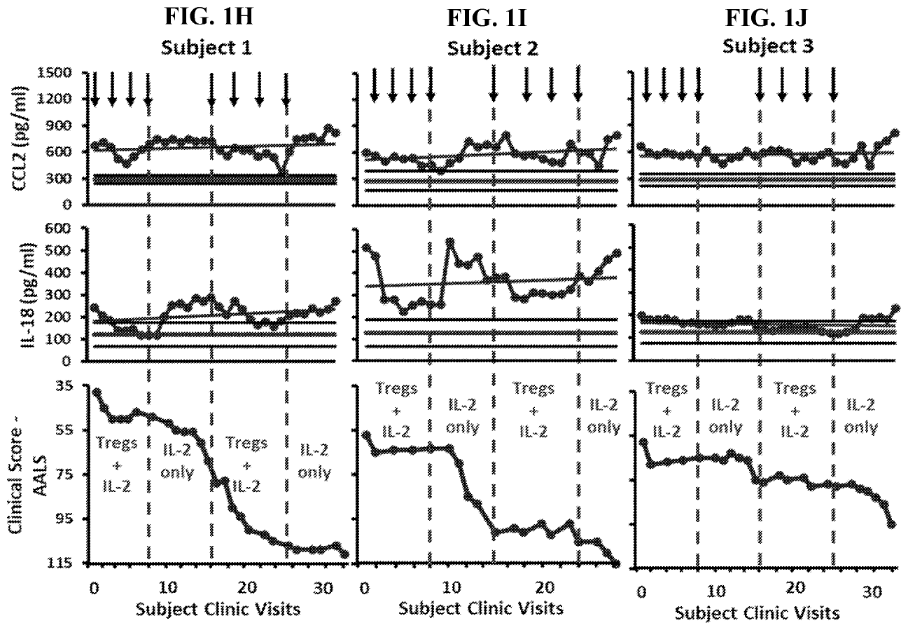

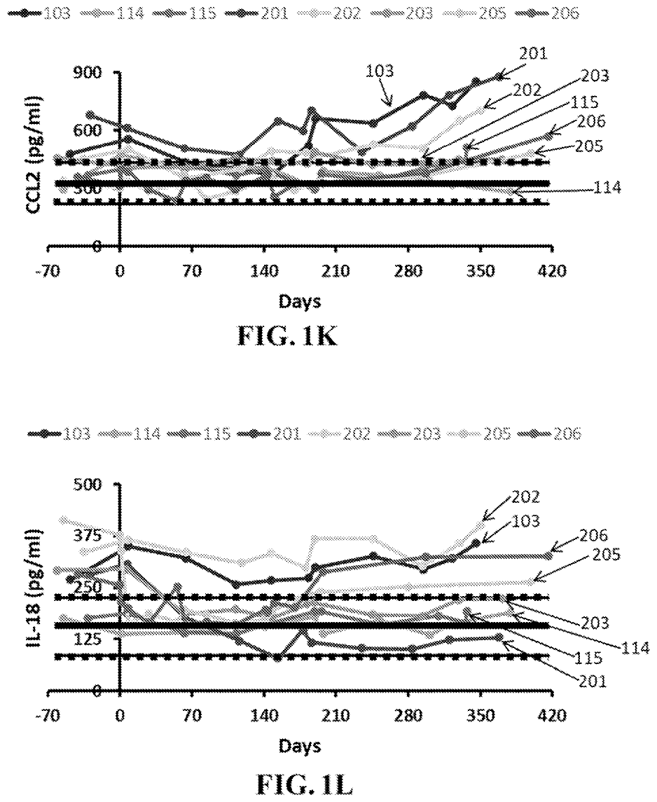

A blinded non-biased assay demonstrated that soluble CCL2 was elevated in serum of patients with ALS (n=30) compared with serum from age-matched HC (n=10, p<0.001) (FIG. 1A). When separated into rapidly and slowly progressing patients, CCL2 was elevated in rapidly progressing patients (n=14) compared with slowly progressing patients (n=16) (p=0.004) or HC (p<0.001) [F(2, 37)=16.43, p<0.001] and CCL2 was elevated in slowly progressing patients compared with HC (p=0.003) (FIG. 1B). IL-18 was elevated in rapidly progressing patients compared with slowly progressing patients or HC (p<0.001) (FIG. 1C). FIG. 1D shows that IL-18 was elevated in rapidly progressing patients compared with slowly progressing patients (p=0.004) or HC (p<0.001) [F(2, 38)=12.19, p<0.001] and IL-18 was elevated in slowly progressing patients compared with HC (p=0.04). CCL2 and IL-18 correlated with disease progression (r=0.554, p=0.001 and r=0.474, p=0.008, respectively) but did not correlate with each other (FIGS. 1E-1G).

Phase I Treg IL-2 trial. Treg/IL-2 infusions suppressed CCL2 and IL-18 levels with a concomitant increase in both when not on therapy but still receiving IL-2 in two of the three phase I subjects (FIGS. 1H and 1I). During the six-month Treg “washout” period, when subjects were receiving only IL-2, CCL2 and IL-18 levels rose toward the baseline level with a coinciding deterioration of the Appel ALS clinical score (AALS). With a second round of infusions, administered every four weeks for a total of 4 infusions, the therapy suppressed CCL2 and IL-18 levels and the subjects' clinical statuses stabilized. At the end of the study when Treg infusions ceased and only IL-2 was continued, CCL2 and IL-18 levels gradually increased again. The stabilization and deterioration of the subjects' clinical statuses mirrored the decline and rise of serum CCL2 and IL-18 levels. The third subject in the study had increased levels of CCL2 that was refractory to Treg/IL-2 therapy; however, IL-18 was not elevated in subject 3 throughout the study (FIG. 1J). Like the other two subjects, subject three's progression stabilized when receiving Treg/IL-2 therapy but progressed again when Treg therapy was stopped and only IL-2 was administered.

Phase IIA Treg/IL-2 trial. Treg/IL-2 combination therapy suppressed CCL2 levels in five of the eight phase IIA subjects. Three phase IIA subjects were progressing rapidly and did not respond to therapy (FIG. 1K). Specifically, Subject Nos. 103, 201, and 202 had CCL-2 levels that did not respond to Treg/IL-2 therapy whereas Subject Nos. 114, 115, 203, 205, and 206 had CCL-2 levels that did respond to Treg/IL-2 therapy. Treg/IL-2 combination therapy suppressed IL-18 levels in six of the eight phase IIA subjects (FIG. 1L). Specifically, Subject Nos. 114, 115, 201, 203, 205, and 206 demonstrated IL-18 levels that responded to Treg/IL-2 therapy whereas Subject Nos. 103 and 202 had IL-18 levels that did not respond to Treg/IL-2 therapy. IL-18 levels in one rapidly progressing phase IIA subject (Subject No. 201) was suppressed (FIG. 1L).

### 7.3.2 OS Analytes

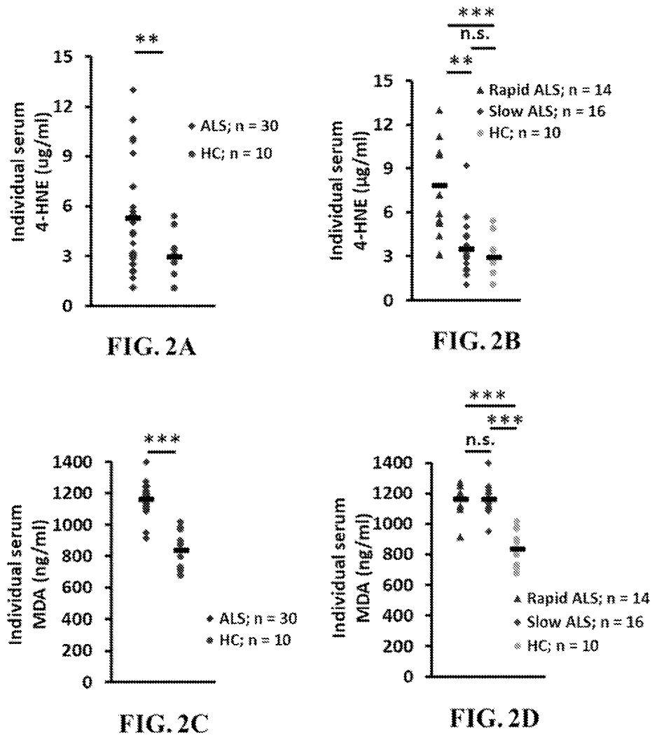

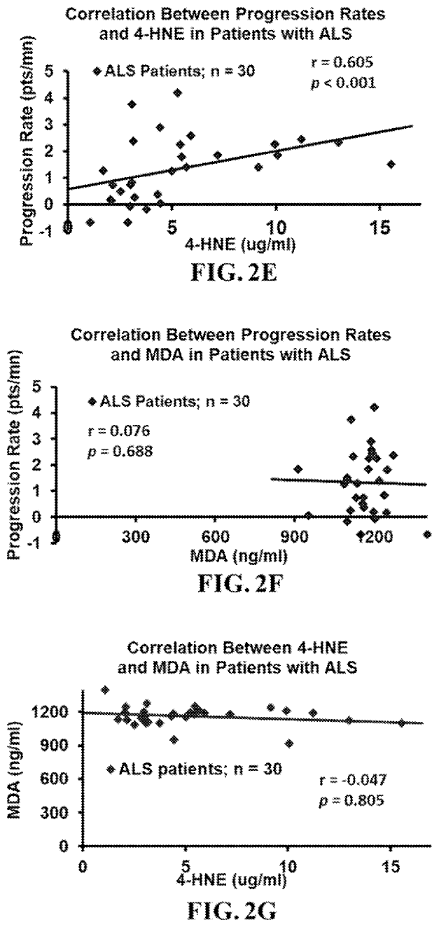

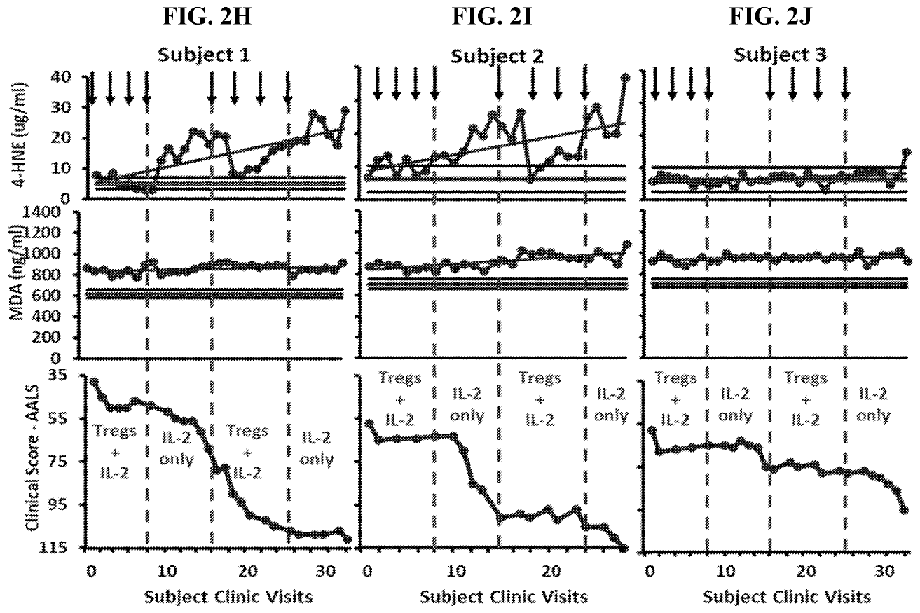

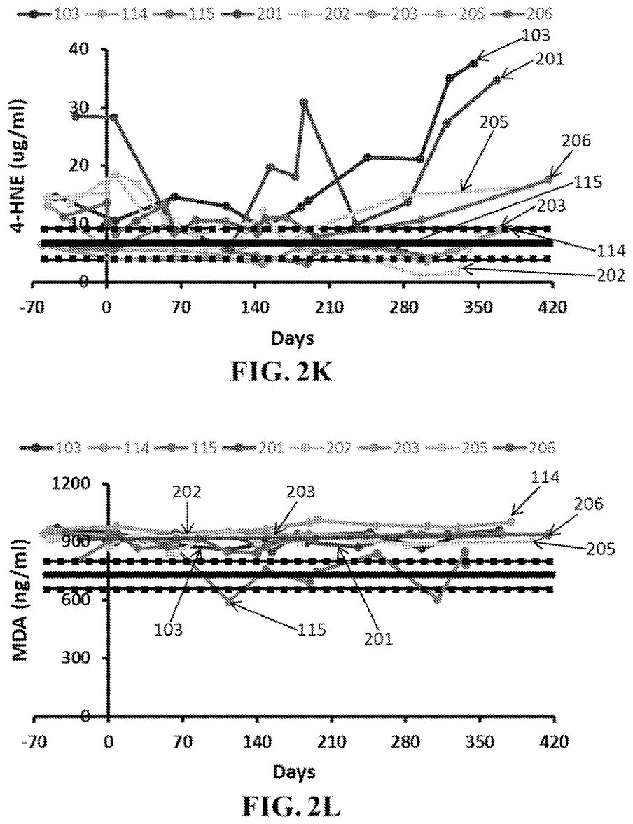

One marker of OS, 4-HNE, was elevated in patients compared with HC (p=0.006). (FIG. 2A). 4-HNE was elevated in rapidly progressing patients compared with slowly progressing patients (p=0.003) or HC (p<0.001) [F(2, 37)=12.05, p<0.001] whereas 4-HNE was not elevated in slowly progressing patients compared with HC (FIG. 2B). A second marker of OS, MDA, was elevated in patients compared with HC (p<0.001) (FIG. 2C). MDA levels were elevated and comparable in both rapidly and slowly progressing patients and levels in both were elevated compared with HC (p<0.001) [F(2, 37)=38.74, p<0.001] (FIG. 2D). FIGS. 2E-2G show that 4-HNE positively correlated with disease progression (r=0.605, p<0.001), but MDA levels did not correlate with progression (r=0.076, p=0.688). 4-HNE and MDA did not correlate with each other (r=−0.047, p=0.805).

Phase I Treg/II-2 trial. Treg infusions suppressed serum 4-HNE levels initially, but 4-HNE levels then rose during the “washout” period when not on Treg therapy in two of the three phase I subjects (FIGS. 2H-2I). Subject three did not have elevated 4-HNE levels throughout the study (FIG. 2J). MDA was longitudinally elevated in the serum of all three subjects throughout the study and the levels were not altered by Treg therapy (FIGS. 2H-2J).

Phase IIA Treg/IL-2 trial. 4-HNE levels were elevated in seven of eight phase IIA subjects and decreased in response to Treg/IL-2 therapy in five of eight subjects (FIG. 2K). Specifically, Subject Nos. 103, 114, 115, 202, 203, 205, and 206 demonstrated elevated 4-HNE levels but only Subject Nos. 114, 115, 202, 205, and 206 demonstrated 4-HNE levels that decreased in response to Treg/IL-2 therapy. MDA was elevated in seven of the eight phase IIA subjects and did not respond to Treg/IL-2 therapy (FIG. 2L). Specifically, Subject Nos. 103, 114, 201, 202, 303, 205, and 206 had MDA levels that were elevated and did not respond to Treg/IL-2 therapy.

### 7.3.3 Neuronal Structural Integrity Analytes

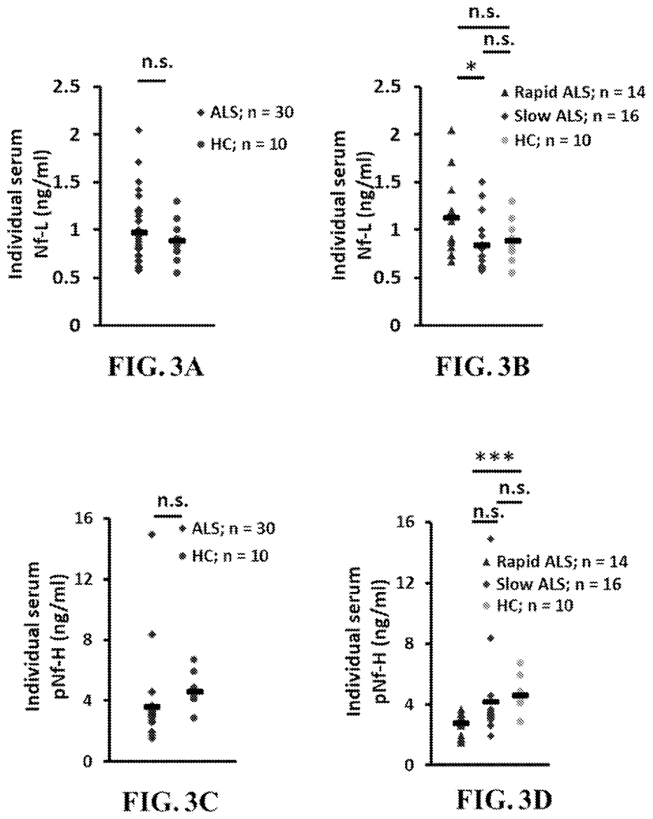

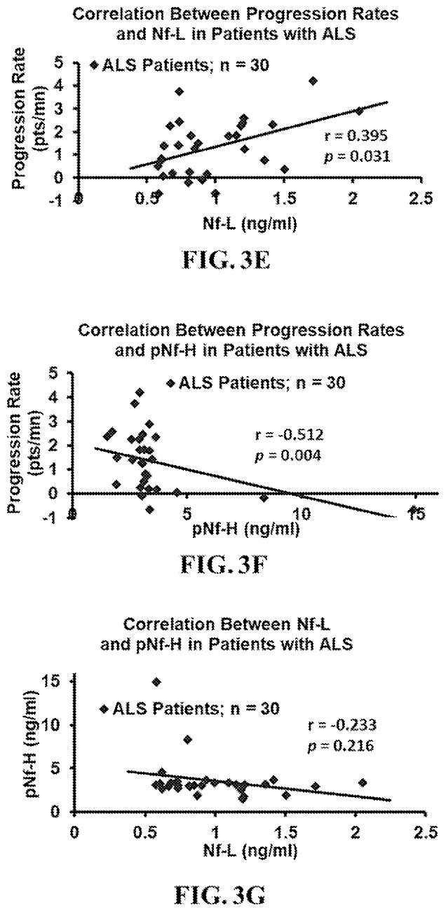

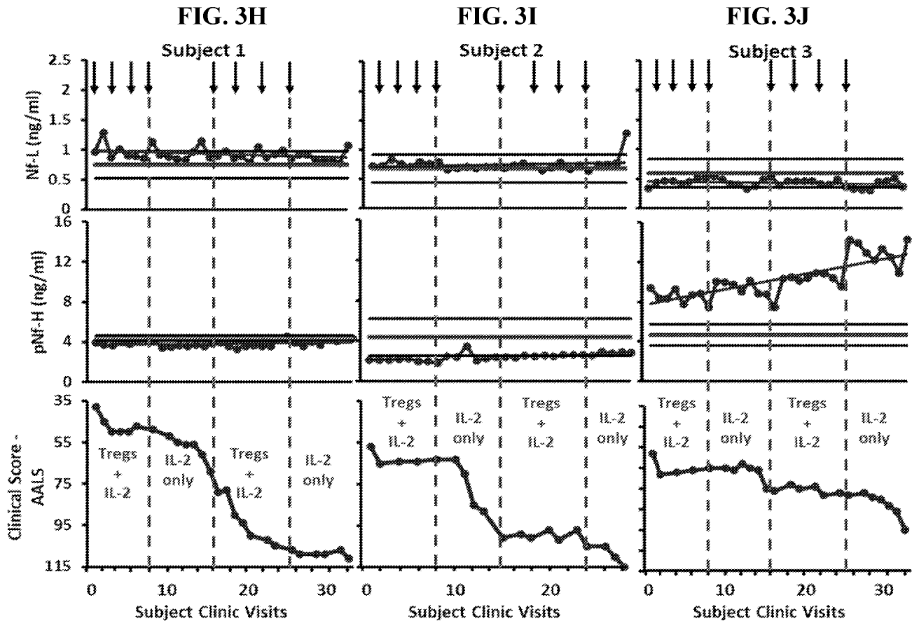

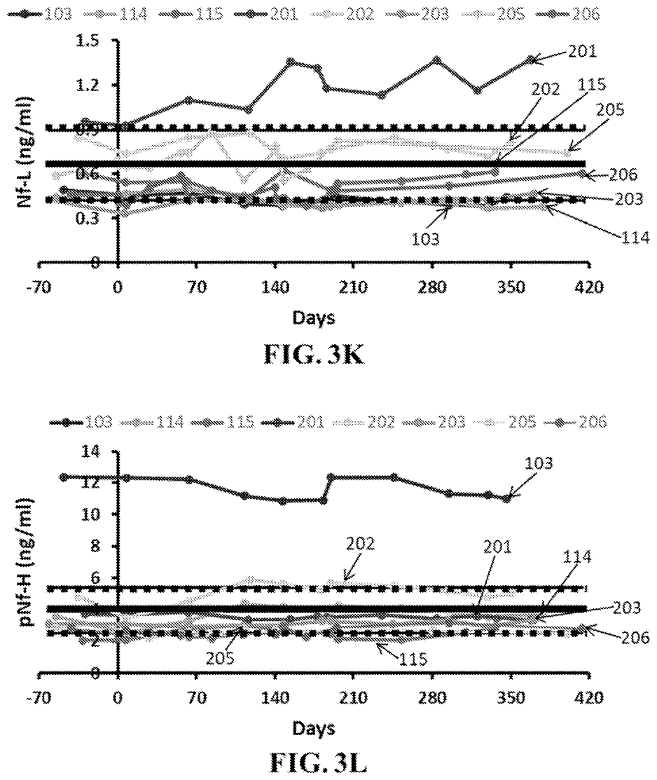

A pathological marker of ALS, Nf-L, was not elevated in the serum of studied cross-sectional patients, but was elevated in rapidly progressing patients compared with slowly progressing patients (p=0.038) [F(2, 37)=3.28, p=0.049] (FIG. 3A). Nf-L was not elevated in both rapidly and slowly progressing patients compared with HC (FIG. 3B). FIGS. 3C-3D show that pNf-H was not elevated in cross-sectionally studied patients compared with HC, but was decreased in rapidly progressing patients compared with HC (p<0.001) [F(2, 37)=2.58, p =0.089]; however, there was no difference in pNf-H levels between slowly progressing patients and HC. There was a weak correlation between Nf-L and disease progression (r=0.395, p=0.031), but there was a negative correlation between pNf-H and disease progression (r=- 0.512, p=0.004), and the neurofilaments did not correlate with each other (r=−0.233, p=0.216) (FIGS. 3E-3G).

Phase I Treg/IL-2 trial. Nf-L and pNf-H levels were not elevated in two of the three longitudinally studied phase I subjects (FIGS. 3H-3I). Only pNf-H was elevated in subject three (FIG. 3I). Neurofilament levels did not respond to Treg/IL-2 therapy in phase I subjects.

Phase IIA Treg/IL-2 trial. Only one of eight subjects in the phase IIA trial had elevated levels of Nf-L (Subject No. 201) whereas a different, individual subject (Subject No. 103) had an elevated level of pNf-H (FIGS. 3K-3L). None of the eleven subjects (phase I and phase II combined) studied longitudinally had elevated levels of both Nf-L and pNf-H. Only three of the twenty-two samples studied longitudinally had elevated levels of either Nf-L or pNf-H (13.6%).

Together, the data provided in Sections 7.3.1, 7.3.2, and 7.3.3 showed that serum soluble inflammatory analytes, CCL2, IL-18, and OS analytes, 4-HNE and MDA, were elevated not only in cross-sectional patient samples, but also in longitudinal patient samples (the cross-sectional and longitudinal data were complementary). CCL2 and IL-18 were elevated in patients, especially rapidly progressing patients, and these two immune factors positively correlated with disease progression and responded to Treg/IL-2 therapy. Further, 4-HNE and MDA were elevated in patients with ALS, and 4-HNE responded to Treg therapy. Thus, the simultaneously assessed levels of inflammatory and oxidative factors, as demonstrated in the present exemplary method, are suitable markers for monitoring disease progression, therapeutic efficacy or effectiveness and/or properly targeting response to ALS therapy, e.g., clinical trial endpoint responses to ALS therapy, including but not limited to Treg/IL-2 ALS therapy.

### 8. EQUIVALENTS

All patents and publications mentioned in this specification are incorporated herein by reference in their entireties. From the foregoing description, it will be apparent that variations and modifications can be made to the invention described herein to adopt it to various uses and conditions. Such embodiments are also within the scope of the following claims.

What is claimed is:

1. A method of treating amyotrophic lateral sclerosis (ALS) comprising:
(a) collecting a serum sample from a patient diagnosed with ALS before administering a first ALS therapy;
(b) administering the first ALS therapy to the patient;
(c) collecting a serum sample from the patient after administering the first ALS therapy;
(d) measuring concentration of at least one biomarker in the serum samples collected from the patient before and after administering the first ALS therapy, wherein the at least one biomarker is selected from the group consisting of oxidized low-density lipoprotein receptor 1 (OLR1), oxidized low-density lipoprotein (ox-LDL), 4-hydroxynonenal (4-HNE), soluble CD14 (sCD14), lipopolysaccharide binding protein (LBP), C-reactive protein (CRP), interleukin 17F (IL-17F), interleukin 17C (IL-17C), monocyte chemoattractant protein-1 MCP-1 (CCL2), and interleukin-18 (IL-18);
(e) comparing the concentration of the at least one biomarker in the serum sample collected from the patient after administering the first ALS therapy to a baseline, wherein the concentration of the at least one biomarker in the serum sample collected from the patient before administering the first ALS therapy is the baseline; and
(f) administering a second ALS therapy to the patient if the concentration of at least one biomarker after administering the first ALS therapy is at least one-fold different than the baseline.

2. The method of claim 1 further comprising collecting serum samples on a serial basis for the duration of the ALS therapy, wherein the serum samples are collected at least once a week, at least once every two weeks, or at least once a month.

3. The method of claim 1 or claim 2, wherein the second ALS therapy is administered to the patient if the concentration of at least 2, at least 3, at least 4, or at least 5 biomarkers are at least one-fold different than the baseline.

4. The method of any one of claims 1-3, wherein the second ALS therapy is administered to the patient if the concentration of at least one biomarker is at least one-fold higher than the baseline.

5. The method of any one of claims 1-4, wherein the second ALS therapy is administered to the patient if the concentration of at least one biomarker is at least one-fold less than the baseline.

6. The method of any one of claims 1-5, wherein the second ALS therapy is administered to the patient if the concentration of at least one biomarker is at least one-fold higher than the baseline and at least one additional biomarker is at least one-fold lower than the baseline.

7. The method of any one of claims 1-6, wherein the second ALS therapy is administered to the patient if the concentration of CCL2, IL-18, or both are at least one-fold lower than the baseline.

8. The method of claim 7, wherein the second ALS therapy administered to the patient is the same as the first ALS therapy administered.

9. The method of any one of claims 1-6, wherein the second ALS therapy is not administered to the patient if the concentration of CCL2, IL-18, or both are at least one-fold higher than the baseline.

10. The method of any one of claims 5-8, wherein the second ALS therapy administered to the patient is not the same as the first ALS therapy administered if the concentration of CCL2, IL-18, or both are at least one-fold higher than the baseline.

11. The method of any one of claims 1-10, wherein the serum sample is collected from the patient at least 1 day, at least 3 days, at least 5 days, at least 1 week, at least 2 weeks, at least 3 weeks, or at least after 4 weeks of administering the first ALS therapy.

12. The method of any one of claims 1-11, wherein the first ALS therapy is the same as the second ALS therapy administered.

13. The method of any one of claims 1-11, wherein the first ALS therapy is different from the second ALS therapy administered.

14. The method of any one of claims 1-13, wherein the ALS therapy comprises one or more agents that targets inflammation and/or oxidative stress.

15. The method of any one of claims 1-14, wherein the ALS therapy comprises at least one gene therapy, wherein the gene therapy targets one or more mutations in genes SOD1, C9orf72, FUS, or any combination thereof.

16. The method of any one of claims 1-15, wherein the ALS therapy comprises a Treg infusion.

17. The method of any one of claims 1-16, wherein the ALS therapy comprises Relyvrio (AMX0035), edaravone, riluzole, or any combination thereof.

18. The method of any one of claims 1-17, wherein the ALS therapy comprises IL-2.

19. The method of any one of claims 1-18, wherein the ALS therapy comprises IL-2 and Abatacept (CTLA-4 Ig).

20. The method of any one of claims 1-19, wherein the ALS therapy comprises an anti-inflammatory agent.

21. The method of any one of claims 1-20, wherein the ALS therapy comprises an antioxidant agent.

22. The method of any one of claims 1-21, wherein the second ALS therapy comprises an anti-inflammatory agent if the concentration of CCL2, IL-18, or both are at least one-fold higher than the baseline.

23. The method of any one of claims 1-22, wherein the second ALS therapy comprises an anti-antioxidant agent if the concentration of ox-LDL, OLR1 or both are at least one-fold higher than the baseline.

24. The method of any one of claims 1-23, wherein the method further comprises determining an Appel ALS clinical score (AALS) for the patient before and after administering the first ALS therapy.

25. The method of claim 24, wherein the second ALS therapy is administered to the patient if the concentration of at least one biomarker is at least one-fold different than the baseline and the AALS score for the patient the same or lower than the AALS score for the patient before administering the first ALS therapy.

26. The method of any one of claims 1-25, wherein the concentration of the at least one biomarker is determined by enzyme linked-immunosorbent assay (ELISA), protein immunoprecipitation, immunoelectrophoresis, chemical analysis, SDS-PAGE and Western blot analysis, protein immunostaining, electrophoresis analysis, competitive binding assay, functional protein assay, protein microarray, high-performance liquid chromatography (HPLC), mass spectrometry, liquid chromatography-mass spectrometry (LC/MS), capillary electrophoresis (CE)-MS, cytometric bead array analysis, immunonephelometric assays, fluorescence activated cell sorting (FACS) analysis, microscopic analysis, microarray, or any combination thereof.

27. A method for selecting a patient for amyotrophic lateral sclerosis (ALS) therapy, the method comprising:
(a) determining whether a concentration of at least one biomarker in a serum sample collected from a patient diagnosed with or suspected of having ALS is less than, equal to, or greater than, a reference concentration;
(b) wherein the at least one biomarker is selected from the group consisting of oxidized low-density lipoprotein receptor 1 (OLR1), oxidized low-density lipoprotein (ox-LDL), monocyte chemoattractant protein-1 MCP-1 (CCL2), interleukin-18 (IL-18), and any combination thereof, and.
(c) selecting the patient for ALS therapy if at least one biomarker is equal to or less than the reference concentration.

28. The method of claim 27, further comprising administering the ALS therapy to the selected patient.

29. The method of claim 27 or 28, wherein the ALS therapy comprises Relyvrio (AMX0035), edaravone, riluzole, Treg infusion, IL-2, or any combination thereof.

30. The method of any one of claims 27-29, wherein the patient is selected for ALS therapy if at least two or more biomarkers are equal to or less than the reference concentration.

31. The method of claim 30, wherein the at least two or more biomarkers are CCL2 and IL-18.

32. The method of any one of claims 27-31, wherein the patient is naïve to ALS therapy.

33. The method of any one of claims 27-32, wherein the patient has previously received at least one ALS therapy.

34. The method of any one of claims 27-33, wherein the patient has received at least one ALS therapy and is selected for a subsequent administration of the same ALS therapy if at least one biomarker is equal to or less than the reference concentration.

35. The method of claim 34, wherein the ALS therapy is a Treg infusion, IL-2, or a combination thereof.

36. The method of any one of claims 27-35, wherein the reference concentration is an average concentration of the biomarker measured in serum samples collected from at least 10 healthy subjects having the same age, gender, and ethnicity as the patient.

37. The method of any one of claims 27-36, wherein the concentration of the at least one biomarker is determined by enzyme linked-immunosorbent assay (ELISA), protein immunoprecipitation, immunoelectrophoresis, chemical analysis, SDS-PAGE and Western blot analysis, protein immunostaining, electrophoresis analysis, competitive binding assay, functional protein assay, protein microarray, high-performance liquid chromatography (HPLC), mass spectrometry, liquid chromatography-mass spectrometry (LC/MS), capillary electrophoresis (CE)-MS, cytometric bead array analysis, immunonephelometric assays, fluorescence activated cell sorting (FACS) analysis, microscopic analysis, microarray, or any combination thereof.

38. A kit for detecting and quantifying the level of one or more ALS biomarkers in a serum sample obtained from a patient having or suspected of having ALS, wherein the ALS biomarkers are selected from the group consisting of oxidized low-density lipoprotein receptor 1 (OLR1), oxidized low-density lipoprotein (ox-LDL), monocyte chemoattractant protein-1 MCP-1 (CCL2), interleukin-18 (IL-18), and any combination thereof.

39. The kit of claim 38, wherein the kit comprises a solid surface having an antibody or a fragment thereof comprising a binding epitope for the ALS biomarkers OLR1, ox-LDL, IL-17C, CCL2, IL-18, or any combination thereof, and at least one reagent for detecting formation of a biomarker-antibody complex.

40. The kit of claim 38 or claim 39 for use in selecting a patient for an ALS therapy.

41. The kit of any one of claims 38-40 for use in monitoring effectiveness of an ALS therapy, wherein effectiveness can be assessed during the course of or after completing an ALS therapy regimen.

42. A method of treating treatment-responsive ALS, comprising administering to a patient diagnosed with treatment-responsive ALS a therapeutically effective amount of an ALS therapy, wherein the treatment-responsive ALS is characterized as having at least one biomarker equal to or less than a reference concentration,
wherein the at least one biomarker is selected from the group consisting of oxidized low-density lipoprotein receptor 1 (OLR1), oxidized low-density lipoprotein (ox-LDL), monocyte chemoattractant protein-1 MCP-1 (CCL2), and interleukin-18 (IL-18); and,
wherein the reference concentration is an average concentration of the biomarker measured in serum samples collected from at least 10 healthy subjects having the same age, gender, and/or ethnicity as the patient.

43. The method of claim 42, wherein treatment-responsive ALS is characterized as having at least two biomarkers equal to or less than a reference concentration.

44. The method of claim 43, wherein the at least two or more biomarkers are CCL2 and IL-18.

45. The method of any one of claims 42-44, wherein the ALS therapy comprises Relyvrio (AMX0035), edaravone, riluzole, Treg infusion, IL-2, or any combination thereof.

46. The method of claim 45, wherein the ALS therapy is a Treg infusion, IL-2, or a combination thereof.

47. A method of determining whether a patient diagnosed with ALS is indicated as likely to be responsive to ALS treatment, comprising detecting the concentration of at least one biomarker compared to a reference concentration in a serum sample collected from the patient, wherein the at least one biomarker having a concentration equal to or less than the reference concentration indicates that the patient is likely to be responsive to ALS treatment,
wherein the at least one biomarker is selected from the group consisting of oxidized low-density lipoprotein receptor 1 (OLR1), oxidized low-density lipoprotein (ox-LDL), monocyte chemoattractant protein-1 MCP-1 (CCL2), and interleukin-18 (IL-18); and,
wherein the reference concentration is an average concentration of the biomarker measured in serum samples collected from at least 10 healthy subjects having the same age, gender, and/or ethnicity as the patient.

48. The method of claim 47, wherein at least two biomarkers having a concentration equal to or less than a reference concentration indicates that the patient is likely to be responsive to ALS treatment.

49. The method of claim 48, wherein the at least two or more biomarkers are CCL2 and IL-18.

50. The method of any one of claims 47-49, wherein the ALS therapy comprises Relyvrio (AMX0035), edaravone, riluzole, Treg infusion, IL-2, or any combination thereof.

51. The method of claim 50, wherein the ALS therapy is a Treg infusion, IL-2, or a combination thereof.
